SEQUENCE LISTING (61 sequences)

SEQ ID NO: 1 - protein, 19 aa, Artificial Sequence
  Synthetic Polypeptide
XRQVXXWXXXRRXXXKXIN

SEQ ID NO: 2 - protein, 20 aa, Homo sapiens
ERQVKIWFQNRRMKMKKINK

SEQ ID NO: 3 - protein, 20 aa, Artificial Sequence
  Synthetic Polypeptide
XXQVSXWXGXKRIXXKKNIG

SEQ ID NO: 4 - protein, 20 aa, Homo sapiens
VSQVSNWFGNKRIRYKKNIG

SEQ ID NO: 5 - protein, 77 aa, Homo sapiens
NNPAANWLHARSTRKKRCPYTKHQTLELEKEFLFNMYLTRDRRYEVARLLNLTERQVKIWFQNRRMKMKKINKDRAK

SEQ ID NO: 6 - protein, 73 aa, Artificial Sequence
  Synthetic Polypeptide
ARRKRRNFXKQATEXLNEYFYSHLXNPYPSEEAKEELAXKXXXTXSQVSNWFGNKRIRYKKNXGKFQEEAXXY

SEQ ID NO: 7 - protein, 73 aa, Homo sapiens
ARRKRRNFNKQATEILNEYFYSHLSNPYPSEEAKEELAKKCGITVSQVSNWFGNKRIRYKKNIGKFQEEANIY

SEQ ID NO: 8 - protein, 73 aa, Homo sapiens
ARRKRRNFSKQATEVLNEYFYSHLSNPYPSEEAKEELAKKCGITVSQVSNWFGNKRIRYKKNIGKFQEEANIY

SEQ ID NO: 9 - protein, 73 aa, Homo sapiens
ARRKRRNFSKQATEILNEYFYSHLSNPYPSEEAKEELAKKCSITVSQVSNWFGNKRIRYKKNIGKFQEEANLY

SEQ ID NO: 10 - protein, 73 aa, Homo sapiens
ARRKRRNFSKQATEVLNEYFYSHLNNPYPSEEAKEELARKGGLTISQVSNWFGNKRIRYKKNMGKFQEEATIY

SEQ ID NO: 11 - protein, 36 aa, Artificial Sequence
  Synthetic Polypeptide
MATAVGMNIQLLLEAADYLERREREAEHGYASMLPY

SEQ ID NO: 12 - protein, 25 aa, Artificial Sequence
  Synthetic Polypeptide
MVGMNIQLLLEAADYLERREREAEH

SEQ ID NO: 13 - protein, 21 aa, Artificial Sequence
  Synthetic Polypeptide
MVGMNIQLLLEAADYLERRER

SEQ ID NO: 14 - protein, 17 aa, Artificial Sequence
  Synthetic Polypeptide
MVGMNIQLLLEAADYLE

SEQ ID NO: 15 - protein, 18 aa, Artificial Sequence
  Synthetic Polypeptide
MNIQLLLEAADYLERRER

SEQ ID NO: 16 - protein, 14 aa, Artificial Sequence
  Synthetic Polypeptide
MNIQLLLEAADYLE

SEQ ID NO: 17 - protein, 14 aa, Mus musculus
NIQLLLEAADYLER

SEQ ID NO: 18 - protein, 8 aa, Artificial Sequence
  Synthetic Polypeptide
DPKKKRKV

SEQ ID NO: 19 - protein, 7 aa, Artificial Sequence
  Synthetic Polypeptide
PKKKRKV

SEQ ID NO: 20 - protein, 16 aa, Artificial Sequence
  Synthetic Polypeptide
DPKKKRKVDPKKKRKV

SEQ ID NO: 21 - protein, 14 aa, Artificial Sequence
  Synthetic Polypeptide
PKKKRKVPKKKRKV

SEQ ID NO: 22 - protein, 24 aa, Artificial Sequence
  Synthetic Polypeptide
DPKKKRKVDPKKKRKVDPKKKRKV

SEQ ID NO: 23 - protein, 21 aa, Artificial Sequence
  Synthetic Polypeptide
PKKKRKVPKKKRKVPKKKRKV

SEQ ID NO: 24 - protein, 43 aa, Homo sapiens
RTLVTFKDVFVDFTREEWKLLDTAQQIVYRNVMLENYKNLVSL

SEQ ID NO: 25 - protein, 290 aa, Artificial Sequence
  Synthetic Polypeptide
MGSSHHHHHHSSGLVPRGSHMAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRSGRELE

SEQ ID NO: 26 - protein, 764 aa, Bacillus anthracis
MKKRKVLIPLMALSTILVSSTGNLEVIQAEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 27 - protein, 735 aa, Artificial Sequence
  Synthetic Polypeptide
EVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPRFMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 28 - protein, 106 aa, Artificial Sequence
  Synthetic Polypeptide
MATAVGMNIQLLLEAADYLERREREAEHGYASMLPYDPKKKRKVDPKKKRKVDPKKKRKVGGDPERQVKIWFQNRRMKMKKINGDPVSQVSNWFGNKRIRYKKNIG

SEQ ID NO: 29 - protein, 107 aa, Artificial Sequence
  Synthetic Polypeptide
MATAVGMNIQLLLEAADYLERREREAEHGYASMLPYDPKKKRKVDPKKKRKVDPKKKRKVGGDPERQVKIWFQNRRMKMKKINGGDPVSQVSNWFGNKRIRYKKNIG

SEQ ID NO: 30 - protein, 108 aa, Artificial Sequence
  Synthetic Polypeptide
MATAVGMNIQLLLEAADYLERREREAEHGYASMLPYDPKKKRKVDPKKKRKVDPKKKRKVGGDPERQVKIWFQNRRMKMKKINGGGDPVSQVSNWFGNKRIRYKKNIG

SEQ ID NO: 31 - protein, 109 aa, Artificial Sequence
  Synthetic Polypeptide
MATAVGMNIQLLLEAADYLERREREAEHGYASMLPYDPKKKRKVDPKKKRKVDPKKKRKVGGDPERQVKIWFQNRRMKMKKINGGGGDPVSQVSNWFGNKRIRYKKNIG

SEQ ID NO: 32 - protein, 44 aa, Artificial Sequence
  Synthetic Polypeptide
DPERQVKAWFAARRAKMKKINGGDPVSQVSAWFGAKRIAYKKNI

SEQ ID NO: 33 - protein, 46 aa, Artificial Sequence
  Synthetic Polypeptide
DPERQVKAWFAARRAKMKKINGGGDPVSQVSAWFGAKRIAYKKNIG

SEQ ID NO: 34 - protein, 46 aa, Artificial Sequence
  Synthetic Polypeptide
DPERQVKIWFQNRRMKMKKINGGGDPVSQVSNWFGNKRIRYKKNIG

SEQ ID NO: 35 - protein, 108 aa, Artificial Sequence; identical to SEQ ID NO: 30
  Synthetic Polypeptide

SEQ ID NO: 36 - protein, 92 aa, Artificial Sequence
  Synthetic Polypeptide
MATAVGMNIQLLLEAADYLERREREAEHGYASMLPYDPKKKRKVGGDPERQVKIWFQNRRMKMKKINGGGDPVSQVSNWFGNKRIRYKKNIG

SEQ ID NO: 37 - protein, 84 aa, Artificial Sequence
  Synthetic Polypeptide
MATAVGMNIQLLLEAADYLERREREAEHGYASMLPYGGDPERQVKIWFQNRRMKMKKINGGGDPVSQVSNWFGNKRIRYKKNIG

SEQ ID NO: 38 - protein, 99 aa, Artificial Sequence
  Synthetic Polypeptide
MVGMNIQLLLEAADYLERREREAEHGGDPKKKRKVDPKKKRKVDPKKKRKVGGDPERQVKIWFQNRRMKMKKINGGGDPVSQVSNWFGNKRIRYKKNIG

SEQ ID NO: 39 - protein, 95 aa, Artificial Sequence
  Synthetic Polypeptide
MVGMNIQLLLEAADYLERRERGGDPKKKRKVDPKKKRKVDPKKKRKVGGDPERQVKIWFQNRRMKMKKINGGGDPVSQVSNWFGNKRIRYKKNIG

SEQ ID NO: 40 - protein, 91 aa, Artificial Sequence
  Synthetic Polypeptide
MVGMNIQLLLEAADYLEGGDPKKKRKVDPKKKRKVDPKKKRKVGGDPERQVKIWFQNRRMKMKKINGGGDPVSQVSNWFGNKRIRYKKNIG

SEQ ID NO: 41 - protein, 92 aa, Artificial Sequence
  Synthetic Polypeptide
MNIQLLLEAADYLERRERGGDPKKKRKVDPKKKRKVDPKKKRKVGGDPERQVKIWFQNRRMKMKKINGGGDPVSQVSNWFGNKRIRYKKNIG

SEQ ID NO: 42 - protein, 88 aa, Artificial Sequence
  Synthetic Polypeptide
MNIQLLLEAADYLEGGDPKKKRKVDPKKKRKVDPKKKRKVGGDPERQVKIWFQNRRMKMKKINGGGDPVSQVSNWFGNKRIRYKKNIG

SEQ ID NO: 43 - protein, 93 aa, Artificial Sequence
  Synthetic Polypeptide
MADPANIQLLLEAADYLERGGDPKKKRKVDPKKKRKVDPKKKRKVGGDPERQVKIWFQNRRMKMKKINGGGDPVSQVSNWFGNKRIRYKKNIG

SEQ ID NO: 44 - protein, 77 aa, Artificial Sequence
  Synthetic Polypeptide
MADPANIQLLLEAADYLERGGDPKKKRKVGGDPERQVKIWFQNRRMKMKKINGGGDPVSQVSNWFGNKRIRYKKNIG

SEQ ID NO: 45 - protein, 107 aa, Artificial Sequence
  Synthetic Polypeptide
DPERQVKIWFQNRRMKMKKINGGGDPVSQVSNWFGNKRIRYKKNIGGGDPKKKRKVDPKKKRKVDPKKKRKVATAVGMNIQLLLEAADYLERREREAEHGYASMLPY

SEQ ID NO: 46 - protein, 108 aa, Artificial Sequence
  Synthetic Polypeptide
MATAVGMNIQLLLEAADYLERREREAEHGYASMLPYDPKKKRKVDPKKKRKVDPKKKRKVGGDPERQVKAWFAARRAKMKKINGGGDPVSQVSAWFGAKRIAYKKNIG

SEQ ID NO: 47 - protein, 428 aa, Artificial Sequence
  Synthetic Polypeptide
MGSSHHHHHHSSGLVPRGSHMAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRSGRELESGGGGSMATAVGMNIQLLLEAADYLERREREAEHGYASMLPYDPKKKRKVDPKKKRKVDPKKKRKVGGDPERQVKIWFQNRRMKMKKINGGGDPVSQVSNWFGNKRIRYKKNIGGGDYKDHDGDYKDHDIDYKDDDDK

SEQ ID NO: 48 - protein, 404 aa, Artificial Sequence
  Synthetic Polypeptide
MGSSHHHHHHSSGLVPRGSHMAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRSGRELESGGGGSMATAVGMNIQLLLEAADYLERREREAEHGYASMLPYDPKKKRKVDPKKKRKVDPKKKRKVGGDPERQVKIWFQNRRMKMKKINGGGDPVSQVSNWFGNKRIRYKKNIG

SEQ ID NO: 49 - protein, 428 aa, Artificial Sequence
  Synthetic Polypeptide
MGSSHHHHHHSSGLVPRGSHMAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRSGRELESGGGGSMDYKDHDGDYKDHDIDYKDDDDKGGDPERQVKIWFQNRRMKMKKINGGGDPVSQVSNWFGNKRIRYKKNIGGGDPKKKRKVDPKKKRKVDPKKKRKVATAVGMNIQLLLEAADYLERREREAEHGYASMLPY

SEQ ID NO: 50 - protein, 405 aa, Artificial Sequence
  Synthetic Polypeptide
MGSSHHHHHHSSGLVPRGSHMAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRSGRELESGGGGSMADPERQVKIWFQNRRMKMKKINGGGDPVSQVSNWFGNKRIRYKKNIGGGDPKKKRKVDPKKKRKVDPKKKRKVATAVGMNIQLLLEAADYLERREREAEHGYASMLPY

SEQ ID NO: 51 - protein, 454 aa, Artificial Sequence
  Synthetic Polypeptide
MGSSHHHHHHSSGLVPRGSDPKKKRKVDPKKKRKVDPKKKRKVGGHMAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRSGRELESGGGGSMATAVGMNIQLLLEAADYLERREREAEHGYASMLPYDPKKKRKVDPKKKRKVDPKKKRKVGGDPERQVKIWFQNRRMKMKKINGGGDPVSQVSNWFGNKRIRYKKNIGGGDYKDHDGDYKDHDIDYKDDDDK

SEQ ID NO: 52 - protein, 430 aa, Artificial Sequence
  Synthetic Polypeptide
MGSSHHHHHHSSGLVPRGSDPKKKRKVDPKKKRKVDPKKKRKVGGHMAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRSGRELESGGGGSMATAVGMNIQLLLEAADYLERREREAEHGYASMLPYDPKKKRKVDPKKKRKVDPKKKRKVGGDPERQVKIWFQNRRMKMKKINGGGDPVSQVSNWFGNKRIRYKKNIG

SEQ ID NO: 53 - protein, 454 aa, Artificial Sequence
  Synthetic Polypeptide
MGSSHHHHHHSSGLVPRGSDPKKKRKVDPKKKRKVDPKKKRKVGGHMAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRSGRELESGGGGSMDYKDHDGDYKDHDIDYKDDDDKGGDPERQVKIWFQNRRMKMKKINGGGDPVSQVSNWFGNKRIRYKKNIGGGDPKKKRKVDPKKKRKVDPKKKRKVATAVGMNIQLLLEAADYLERREREAEHGYASMLPY

SEQ ID NO: 54 - protein, 431 aa, Artificial Sequence
  Synthetic Polypeptide
MGSSHHHHHHSSGLVPRGSDPKKKRKVDPKKKRKVDPKKKRKVGGHMAGGHGDVGMHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRSGRELESGGGGSMADPERQVKIWFQNRRMKMKKINGGGDPVSQVSNWFGNKRIRYKKNIGGGDPKKKRKVDPKKKRKVDPKKKRKVATAVGMNIQLLLEAADYLERREREAEHGYASMLPY

SEQ ID NO: 55 - protein, 4 aa, Artificial Sequence
  Synthetic Polypeptide
GGGG

SEQ ID NO: 56 - protein, 5 aa, Artificial Sequence
  Synthetic Polypeptide
GGGGG

SEQ ID NO: 57 - protein, 6 aa, Artificial Sequence
  Synthetic Polypeptide
SGGGGS

SEQ ID NO: 58 - protein, 11 aa, Artificial Sequence
  Synthetic Polypeptide
SGGGGSGGGGS

SEQ ID NO: 59 - protein, 11 aa, Human immunodeficiency virus
YGRKKRPQRRR

SEQ ID NO: 60 - protein, 15 aa, Artificial Sequence
  Synthetic Polypeptide
KRPTMRFRYTWNPMK

SEQ ID NO: 61 - protein, 20 aa, Artificial Sequence
  Synthetic Polypeptide
XXQVSNWXGNKRIRXKKNIG

(19) United States
(12) Patent Application Publication
(10) Pub. No.: US 2016/0122405 A1
(43) Pub. Date: May 5, 2016

(54) HOMEODOMAIN FUSION PROTEINS AND USES THEREOF

(71) Applicant: PRESIDENT AND FELLOWS OF HARVARD COLLEGE, Cambridge, MA (US)

(72) Inventors: Rahul Palchaudhuri, Cambridge, MA (US); David T. Scadden, Weston, MA (US); Gregory L. Verdine, Boston, MA (US)

(73) Assignees: President and Fellows of Harvard College, Cambridge, MA (US); The General Hospital Corporation d/b/a Massachusetts General Hospital, Boston, MA (US)

(21) Appl. No.: 14/896,132

(22) PCT Filed: Jun. 6, 2014
(86) PCT No.: PCT/US2014/041338

§ 371 Date: Dec. 4, 2015

Related U.S. Application Data
(60) Provisional application No. 61/832,043, filed on Jun. 6, 2013.

(51) Int. Cl. C07K 14/47

(52) U.S. Cl. CPC C07K 14/4703; C07K 2319/00; C07K 2319/09

(57) ABSTRACT

Provided herein are fusion proteins comprising a homeodomain fusion protein domain and a transcription modulator domain for treatment of various diseases or disorders such as cancer. The homeodomain fusion protein domain binds to a target gene and the transcription modulator domain either activates or represses gene transcription. The present invention also relates to polynucleotides encoding the fusion proteins, vectors comprising the polynucleotides, cells comprising the polynucleotides, vectors, or fusion proteins. Also provided are methods of use and compositions for delivery of the fusion proteins.

### RELATED APPLICATIONS

This application claims priority under 35 U.S.C. §119(e) to U.S. Provisional Patent Application, U.S. Ser. No. 61/832,043, filed Jun. 6, 2013, which is incorporated herein by reference in its entirety.

### GOVERNMENT SUPPORT

This invention was made with government support under HL097748 and HL097794 awarded by the National Institutes of Health. The government has certain rights in the invention.

### BACKGROUND

Acute myeloid leukemia (AML) is the second most common leukemia in children and adults, and a particularly devastating blood cancer with a 5-year survival rate of only 24%.(1) It is estimated that 19,000 people will be diagnosed with AML in the US in 2014, with approximately 10,500 deaths expected this year.(1) Chemotherapy regimens for the bulk of AML patients have remained unchanged for 50 years.(2) While the differentiation-inducing therapy, all-trans-retinoic acid (ATRA), has drastically changed the outcome of a subset (˜10%) of AML patients (3) specifically those with acute promyelocytic leukemia (APML), differentiation therapy is severely lacking for the remaining 90% of AML patients.

Analysis of 6817 genes in AML patient samples revealed the homeodomain protein, HoxA9, as the single most highly correlated gene for poor prognosis.(4) Currently ˜200 homeodomain proteins are known to play a role in human diseases.(5) Homeodomain proteins contain a 60 amino acid helix-turn-helix homeodomain motif that binds to DNA in a sequence-selective manner to regulate gene transcription.(5) In normal hematopoiesis, the expression of HoxA9 is downregulated as cells differentiate and mature.(6, 7) However in 70% of AML cases, HoxA9 together with Meis1, another homeodomain protein, are inappropriately and persistently expressed leading to a block in cell differentiation that enables leukemia progression (FIG. 1B).(8) HoxA9 transduction in murine bone marrow immortalizes myeloid progenitors in culture (9) and results in AML upon transplantation in murine recipients with a latency of 180 days.(10) However, co-transduction of Meis1 (with HoxA9) results in a highly aggressive disease with latency of 30-60 days, highlighting the collaborative nature of Meis1 and HoxA9 in AML progression.(11)

HoxA9 and Meis1, together with PBX (another homeodomain protein), form a DNA-binding complex with transcriptional activating properties leading to the expression of differentiation-blocking genes in AML (see FIG. 1).(12, 13) In vitro and cell-based studies have revealed the DNA recognition sequence of the Hoxa9-PBX complex as TGATTTAT, in which PBX binds to the 5′ TGAT and Hoxa9 binds to 3′ TTAT.(14, 15) The crystal structure of Hoxa9-Pbx1-DNA complex reveals that the DNA-recognition helices in the homeodomains of Hoxa9 and Pbx1 are adjacent to each other, enabling contiguous DNA site-recognition.(16) Meis1 does not appear to play a role in DNA-binding but is recruited to the complex by interactions with Hoxa9 and Pbx1, and the transcription-activating domain (TAD) of Meis1 enables transcription of differentiation-blocking genes.(11, 17)

Active transcriptional repression at Hoxa9-Pbx1 genomic DNA binding sites is expected to counter endogenous Hoxa9/PBX/Meis1 activity and enable AML differentiation. Hoxa9 and other Hox proteins are also misregulated in various other cancers and contribute to their progression.(18) However, current DNA-targeting technologies preclude the creation of therapies capable of transiently modulating transcription. For example, zinc finger and transcription activator-like effector (TALE) proteins are too large to create effective cell-permeable versions. Therefore, AML remains a largely untreatable and deadly disease, and there remains a need for novel differentiation-based therapy for AML.

### SUMMARY OF THE INVENTION

Provided herein are fusion proteins useful as sequence-specific DNA-targeting therapeutics in various diseases and disorders. For example, the fusion proteins are useful for the treatment of cancers such as acute myeloid leukemia (AML).

In one aspect, provided herein are fusion proteins comprising a homeodomain fusion protein (HFP) domain and a transcription modulator (TM) domain, wherein the homeodomain fusion protein domain comprises a first homeodomain and a second homeodomain. In one aspect, provided herein are fusion proteins comprising a homeodomain fusion protein (HFP) domain, a nuclear localization sequence (NLS) domain, and a transcription modulator (TM) domain, wherein the homeodomain fusion protein domain comprises a first homeodomain and a second homeodomain.

In certain embodiments, the fusion protein comprises a HoxA9 homeodomain comprising the sequence: X₁RQVX₅X₆WX₈X₉X₁₀RRX₁₃X₁₄X₁₅KX₁₇IN (SEQ ID NO: 1), wherein X₁, X₅, X₆, X₈, X₉, X₁₀, X₁₃, X₁₄, X₁₅, X₁₇ are as defined herein.

In certain embodiments, the fusion protein comprises a PBX homeodomain comprising the sequence: X₁X₂QVSX₆WX₈GX₁₀KRIX₁₄X₁₅KKNIG (SEQ ID NO: 3), wherein X₁, X₂, X₆, X₈, X₁₀, X₁₄, X₁₅ are as defined herein.

In certain embodiments, the fusion protein comprises an HFP domain comprising a first homeodomain that is HoxA9 of SEQ ID NO: 5 or a variant thereof, and a second homeodomain that is PBX of SEQ ID NO: 6 or a variant thereof.

In certain embodiments, the fusion protein comprises an HFP domain comprising a first homeodomain comprising a sequence that is at least about 80% homologous or identical to HoxA9 of SEQ ID NO: 5, and a second homeodomain comprising a sequence that is at least about 80% homologous or identical to PBX of SEQ ID NO: 6.

In certain embodiments, the fusion protein comprises a transcription modulator domain that is a transcription repressor domain or a transcription activator domain. In certain embodiments, the fusion protein comprises at least one homeodomains or transcription modulator domains that is stapled or stitched. In certain embodiments, the fusion protein comprises polyglycine linkers. In certain embodiments, the fusion protein comprises an alpha-helix nucleating motif. In certain embodiments, the fusion protein comprises comprises an anthrax toxin lethal factor. In certain embodiments, the fusion protein comprises a cell-penetrating peptide.

In another aspect, provided herein are polynucleotides encoding the fusion proteins described herein; vectors comprising the polynucleotides; cells comprising a polynucleotides, vectors, and/or fusion protein.

In still another aspect, provided herein are compositions comprising a pore-forming toxin unit and the fusion protein described herein. In certain embodiments, the pore-forming toxin domain is a protective antigen and the fusion protein comprises a complementary toxin domain such as LF_{N}. The protective-antigen can be wild-type protective-antigen or a mutant protective-antigen such as a protective-antigen comprising the mutations N682A and D683A. The mutant protective-antigen can be fused to a cell-targeting domain such as antibody. In certain embodiments, the antibody is a scFv that is specific to CD33.

In a further aspect, provided are pharmaceutical composition comprising the compositions described herein and a pharmaceutically acceptable carrier or excipient.

The fuson proteins described herein are useful in methods and systems for treating a disease or disorder, the method comprising administration of the inventive fusion proteins to a subject in need thereof. The inventive concepts herein are useful to treat a disease or disorder is associated with aberrant Hox activity such as cancer and specifically, acute myeloid leukemia (AML).

### DEFINITIONS

A “homeodomain” is a DNA-binding protein domain which can bind to target sequences in genes and regulate their expression during development. Homeobox (HOX) genes contain a highly conserved nucleotide sequence of about 180 by which encodes a homeodomain of about 60 amino acids. The homeodomains typically bind close to the transcription start site on the targets or within a promoter region for the target gene. Exemplary target genes include CD34, which is a marker of primitive hemaotpoietic progenitors and FoxP1, which is important in hematopoietic stem cell maintenance. The clustered HOX genes are key developmental regulators and are highly conserved throughout evolution. The homeotic Hox proteins which they encode function as transcription factors to control axial patterning by regulating the transcription of subordinate downstream genes, e.g., developmental genes. Hox was shown to preferentially bind to the consensus sequence TNAT, wherein N can be A, T, G, or c. Various exemplary Hox proteins can be found in Shah & Sukumar (2010) Nat. Rev. Cancer. 10(5):361-71. Non-limiting examples of Hox proteins include HoxA proteins, HoxB proteins, HoxC proteins, and Hox D proteins. Specific non-limiting examples include HoxA1-A13, HoxB1-B13, HoxC1-C13, and HoxD1-D13. Over 206 homeodomain proteins have been implicated in human diseases (see research.nhgri.nih.gov/homeodomain/?mode=like&view=disorders&sortby=ENTREZ_GEN E_SYMBOL). In certain embodiments, the fusion proteins provided herein comprise a homeodomain of a Hox protein. In certain embodiments, the fusion proteins provided herein comprise the HoxA9 homeodomain sequence of ERQVKIWFQNRRMKMKKINK (SEQ ID NO: 2). Human HoxA9 can be found under the identification number P31269 at www.uniprot.org. In the foregoing sequence, DNA backbone contact residues are single-underlined, and DNA base contact residues are double-underlined. HoxA9 is a posterior-regulating Hox protein required for proper limb development in mammals and is implicated as a factor in the induction of Acute Myeloid Leukemia (AML).

The term “PBX” refers to pre-B cell leukemia transcription factors (PBXs). PBXs act as cofactors in the transcriptional regulation mediated by Homeobox (Hox) proteins during embryonic development and cellular differentition. PBXs are in a group called three amino acid loop extension (TALE) homeobox proteins that are highly conserved transcription regulators. PBX proteins are important regulatory proteins that control gene expression during development by interacting cooperatively with Hox proteins to bind to the target DNA. PBX binds to the consensus sequence TGAT. Exemplary PBXs include, but are not limited to, Pbx1, Pbx2, Pbx3, and Pbx4. The full amino acid sequence for human Pbx1, Pbx2, Pbx3, Pbx4 can be found under the identification numbers P40424, P40425, P40426, and Q9BYU1, respectively, at www.uniprot.org. The Pbx members Pbx1, Pbx2, and Pbx3 have closely related sequences. As used herein, a “truncated PBX homeodomain” refers to the sequence: VSQVSNWFGNKRIRYKKNIG (SEQ ID NO: 4), which is common to Pbx1, Pbx2, and Pbx3. In the foregoing sequence, DNA backbone contact residues are single-underlined, and DNA base contact residues are double-underlined. As used herein, a PBX homeodomain or a full-length PBX homeodomain refers to the sequence: ARRKRRNFX₉ KQATEX₁₅ LNEYFYSHLX₂₅NPYPSEEAKEELAX₃₉KX₄₁X₄₂X₄₃TX₄₅SQVS NWFGNKRIRYKKNX₆₃GKFQEEAX₇₁X₇₂ Y (SEQ ID NO: 6), wherein X₉ is N, X₁₅ is I, X₂₅ is S, X₃₉ is K, X₄₁ is C, X₄₂ is G, X₄₃ is I, X₄₅ is V, X₆₃ is I, X₇₁ is N, and X₇₂ is I; wherein X₉ is S, X₁₅ is V, X₂₅ is S, X₃₉ is K, X₄₁ is C, X₄₂ is G, X₄₃ is I, X₄₅ is V, X₆₃ is I, X₇₁ is N, and X₇₂ is I; wherein X₉ is S, X₁₅ is I, X₂₅ is S, X₃₉ is K, X₄₁ is C, X₄₂ is S, X₄₃ is I, X₄₅ is V, X₆₃ is I, X₇₁ is N, and X₇₂ is L; or wherein X₉ is 5, X₁₅ is V, X₂₅ is N, X₃₉ is R, X₄₁ is G, X₄₂ is G, X₄₃ is L, X₄₅ is I, X₆₃ is M, X₇₁ is Y, and X₇₂ is I.

In certain embodiments, the fusion protein provided herein comprises the homeodomain of the Pbx proteins. In certain embodiments, the fusion protein provided herein comprises a truncated sequence of the homeodomain of a Pbx protein. In certain embodiments, the fusion protein provided herein comprises the pre-B-cell leukemia transcription factor 1 (Pbx1) homeodomain.

“E2A” is a member of the E-protein family of basic helix-loop-helix (bHLH) proteins. The E2A gene encodes 2 E proteins (“E2A proteins”), E12 and E47, which are generated by differential splicing of the exon encoding the DNA binding and dimerization domain. E2A proteins are central regulators in early B cell differentiation and are required for proper B cell development and initiation of immunoglobulin gene rearrangements. The chimeric oncoprotein E2A-PBX1 is expressed as a result of the t(1;19) chromosomal translocation and gives rise to B cell-acute lymphoblastic leukemia (ALL). The E2A-Pbx1 chimeric transcription factor contains the N-terminal transactivation domain of E2A (TCF3) fused to the C-terminal DNA-binding homeodomain of PBX1. Fusion proteins useful as a B-cell therapeutic would include two PBX homeodomains as the homeodomain fusion protein domain linked to a transcription repression domain.

The term “polyglycine” is defined to mean at least one glycine or at least two, three, four, or five consecutive glycines. For example, the polyglycine linker can be G, GG, GGG, GGGG (SEQ ID NO: 55), or GGGGG (SEQ ID NO: 56). The polyglycine linker can also include other amino acids. For example, a polyglycine linker may include a combination of glycines and serines. For example, S(G)xS(G)yS, wherein x and y can be an integer between 1-5. In certain embodiments, the polyglycine linker is (SGGGGS)ₙ (SEQ ID NO: 57), wherein n is 1 to 4. In certain embodiments, n is 1. In certain embodiments, n is 2. In certain embodiments, n is 3. In certain embodiments, n is 4. In certain embodiments, the polyglycine linker is SGGGGS (SEQ ID NO: 57) or SGGGGSGGGGS (SEQ ID NO: 58).

The terms “variant” or “mutant” are used interchangeably and means a polypeptide based on the wild-type parent polypeptide comprising at least one alteration, i.e., a substitution, insertion, and/or deletion, at one or more positions of the polypeptide or the polynucleotide encoding the polypeptide. Variants include truncated forms of a polypeptide wherein one or more amino acids are removed from either or both the N-terminal side or C-terminal side. A substitution means a replacement of an amino acid occupying a position with a different amino acid; a deletion means removal of an amino acid occupying a position; and an insertion means adding 1-3 amino acids adjacent to an amino acid occupying a position. Variants include those with homologous mutations in another related homeodomain protein that corresponds to the amino acid mutations specifically listed herein that is expected to have a similar effect to a substantially similar mutation in another homeodomain protein. One of skill in the art can easily locate a homologous residue in their desired homeodomain protein by performing an alignment of the desired homeodomain protein with a homeodomain protein sequence using a computer program such as Clusta1W. Examples of homologous mutations include the mutations made in the Examples set forth in this application. The terms variant or mutant also refers to a polynucleotide variant encoding a polypeptide variant described herein. The polynucleotide variant encompasses all forms of mutations including deletions, insertions, and point mutations in the coding sequence. The polynucleotides provided herein may be DNA or RNA.

The term “homologous,” as used herein, is an art-understood term that refers to nucleic acids or proteins that are highly related at the level of nucleotide or amino acid sequence. Nucleic acids or proteins that are homologous to each other are termed homologues. Homologous may refer to the degree of sequence similarity between two sequences (i.e., nucleotide or amino acid sequence). The homology percentage figures referred to herein reflect the maximal homology possible between two sequences, i.e., the percent homology when the two sequences are so aligned as to have the greatest number of matched (homologous) positions. Homology can be readily calculated by known methods such as those described in: Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. Methods commonly employed to determine homology between sequences include, but are not limited to, those disclosed in Carillo, H., and Lipman, D., SIAM J Applied Math., 48:1073 (1988), incorporated herein by reference. Techniques for determining homology are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and PASTA Atschul, S. F. et al., J Molec. Biol., 215, 403 (1990)).

The term “identity” refers to the overall relatedness between nucleic acids (e.g., DNA and/or RNA) or between proteins. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM 120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Atschul, S. F. et al., J. Molec. Biol., 215, 403 (1990)).

As used herein, the term “protein” refers to a polymer of at least two amino acids linked to one another by peptide bonds. The terms, “protein”, “polypeptides”, and “peptides” are used interchangeably herein. Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example, linked by one or more disulfide bonds or associated by other means. A polypeptide may refer to an individual peptide or a collection of polypeptides. Polypeptides may contain L-amino acids, D-amino acids, or both and may contain any of a variety of amino acid modifications or analogs known in the art. Useful modifications include, e.g., addition of a chemical entity such as a carbohydrate group, a phosphate group, a farnesyl group, an isofarnesyl group, a fatty acid group, an amide group, a terminal acetyl group, a linker for conjugation, functionalization, or other modification (e.g., alpha amidation), etc. In a preferred embodiment, the modifications of the peptide lead to a more stable peptide (e.g., greater half-life in vivo). These modifications may include cyclization of the peptide, the incorporation of D-amino acids, stapling, stitching, etc. None of the modifications should substantially interfere with the desired biological activity of the peptide. In certain embodiments, the modifications of the peptide lead to a more biologically active peptide. In certain embodiments, polypeptides may comprise natural amino acids, non-natural amino acids (i.e., amino acids that do not occur in nature but that can be incorporated into a peptide chain), synthetic amino acids, amino acid analogs, and combinations thereof. A polypeptide may be just a fragment of a naturally occurring protein. A polypeptide may be naturally occurring, recombinant, synthetic, or any combination thereof.

As used herein, “cross-linking” peptides refers to either covalently cross-linking peptides or non-covalently cross-linking peptides. In certain embodiments, the peptides are covalently associated. Covalent interaction is when two peptides are covalently connected through a linker group such as a natural or non-natural amino acid side chain. In other embodiments, the peptides are non-covalently associated. Non-covalent interactions include hydrogen bonding, van der Waals interactions, hydrophobic interactions, magnetic interactions, and electrostatic interactions. The peptides may also comprise natural or non-natural amino acids capable of cross-linking the peptide with another peptide.

A “stapled” or “stitched” protein means that the protein underwent peptide stapling or stitching. “Peptide stapling” is one method for crosslinking within a peptide (intrapeptide) or between different peptides (interpeptide). Peptide stapling describes a synthetic methodology wherein two olefin-containing sidechains present in a peptide or different peptides are covalently joined (“stapled”) using a ring-closing metathesis (RCM) reaction to form a crosslink (see, the cover art for J. Org. Chem. (2001) vol. 66, issue 16 describing metathesis-based crosslinking of alpha-helical peptides; Blackwell et al.; Angew Chem. Int. Ed. (1994) 37:3281; and U.S. Pat. No. 7,192,713). “Peptide stitching” involves multiple “stapling” events in a single polypeptide chain to provide a multiply stapled (also known as “stitched”) polypeptide (see, for example, Walensky et al., Science (2004) 305:1466-1470; U.S. Pat. No. 8,592,377; U.S. Pat. No. 7,192,713; U.S. Patent Application Publication No. 2006/0008848; U.S. Patent Application Publication No. 2012/0270800; International Publication No. WO 2008/121767, and International Publication No. WO 2011/008260). Stapling of a peptide using all-hydrocarbon crosslinks has been shown to help maintain its native conformation and/or secondary structure, particularly under physiologically relevant disorders (see Schafmiester et al., J. Am. Chem. Soc. (2000) 122:5891-5892; Walensky et al., Science (2004) 305:1466-1470). In certain embodiments, the non-natural amino acids found in the fusion proteins described herein comprise a side chain capable of being covalently joined using olefin moieties (i.e., “stapled together”) using a cross-linking reaction such as a ring-closing metathesis (RCM) reaction.

The term “antibody” refers to an immunoglobulin (e.g., IgG, IgM, IgA, IgE, IgD, etc.). The basic functional unit of each antibody is an immunoglobulin (Ig) monomer (containing only one immunoglobulin (“Ig”) unit). Included within this definition are monoclonal antibodies, chimeric antibodies, recombinant antibodies, and humanized antibodies. In one embodiment, the antibodies are monoclonal antibodies produced by hybridoma cells. In particular, the invention contemplates antibody fragments that contain the idiotype (“antigen -binding fragment”) of the antibody molecule. For example, such fragments include, but are not limited to, the Fab region, F(ab′)2 fragment, pFc′ fragment, and Fab′ fragments.

The “Fab region” and “fragment, antigen binding region,” interchangeably refer to portion of the antibody arms of the immunoglobulin “Y” that function in binding antigen. The Fab region is composed of one constant and one variable domain from each heavy and light chain of the antibody. Methods are known in the art for the construction of Fab expression libraries (Huse et al., Science, 246: 1275-1281 (1989)) to allow rapid and easy identification of monoclonal Fab fragments with the desired specificity. In another embodiment, Fc and Fab fragments can be generated by using the enzyme papain to cleave an immunoglobulin monomer into two Fab fragments and an Fc fragment. The enzyme pepsin cleaves below the hinge region, so a “F(ab′)2 fragment” and a “pFc′ fragment” is formed. The F(ab')2 fragment can be split into two “Fab′ fragments” by mild reduction.

The invention also contemplates a “single-chain antibody” fragment, i.e., an amino acid sequence having at least one of the variable or complementarity determining regions (CDRs) of the whole antibody, and lacking some or all of the constant domains of the antibody. These constant domains are not necessary for antigen binding, but constitute a major portion of the structure of whole antibodies. Single-chain antibody fragments are smaller than whole antibodies and may therefore have greater capillary permeability than whole antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely to provoke an immune response in a recipient than whole antibodies. Techniques for the production of single-chain antibodies are known (U.S. Pat. No. 4,946,778). The variable regions of the heavy and light chains can be fused together to form a “single-chain variable fragment” (“scFv fragment”), which is only half the size of the Fab fragment, yet retains the original specificity of the parent immunoglobulin.

The “Fc” and “Fragment, crystallizable” region interchangeably refer to portion of the base of the immunoglobulin “Y” that function in role in modulating immune cell activity. The Fc region is composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins. By doing this, it mediates different physiological effects including opsonization, cell lysis, and degranulation of mast cells, basophils and eosinophils. In an experimental setting, Fc and Fab fragments can be generated in the laboratory by cleaving an immunoglobulin monomer with the enzyme papain into two Fab fragments and an Fc fragment.

As used herein the term “comprising ” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

### BRIEF DESCRIPTION OF THE FIGURES

The accompanying drawings are not intended to be drawn to scale. In the Drawings, for purposes of clarity, not every component may be labeled in every drawing.

FIG. 1A illustrates the HoxA9-PBX-Meis1 complex binding to DNA and transcribes differentiation-blocking genes critical to AML. FIG. 1B illustrates an exemplary repressor fusion protein comprising SID-3xNLS-HoxA9-3XGly-PBX.

FIG. 2 illustrates screening of fusion proteins using yeast surface display library by fluorescence activated cell sorting.

FIG. 3 shows that fusion proteins S1-S4 repressors elevate the mRNA levels of certain myeloid cell differentiation-specific genes. GFP is the first bar, Si is the second bar, S2 is the hird bar, S3 is the fourth bar, and S4 is the fifth bar for each gene on the x-axis.

FIG. 4 shows the growth phenotype of cells containing the fusion proteins.

FIG. 5A shows data from quantitative PCR (QPCR) of mutants and wild-type constructs on day 17 in Hoxa9-Meis1 cells. GFP is the first bar, SID is the second bar, S2 is the third bar, S3 is the fourth bar, S2M is the fifth bar, S3M is the sixth bar, VP64 is the seventh bar, V1 is the eighth bar, V2 is the ninth bar, V3 is the tenth bar, and V4 is the eleventh bar for each construct on the x axis. FIG. 5B is an expanded view of the QPCR data for the S100A8 and Meis1A markers.

FIG. 6 shows data from QPCR of mRNA levels for direct Hoxa9 or Meis1 targets in Hoxa9/Meis1 Cells, 17 days after transduction. *indicates that the data is statistically significant (p<0.05). GFP control is the first bar, S3 is the second bar, and S3M is the third bar for each target on the x-axis. The figure shows that the S3 repressor suppresses transcripts of Hoxa9 target genes while the S3 mutant does not (statistically significant (p<0.05). Hoxa9-Meis1 murine AML cells were transduced with retroviral vectors comprising polynucleotides encoding the fusion protein S3 (“repressor”) or S3 mutant (“mutant”). Total RNA was harvested from Hoxa9-Meis1 murine AML cells expressing MSCV IRES GFP vector-only, S3, or S3 mutant and analyzed for repression of Hoxa9-specific target genes by QPCR. Target HoxA9 genes were identified using published Chip-Seq data for HoxA9.

FIG. 7 shows the cell surface markers on day 30 after cell transduction.

FIG. 8 shows activator constructs Meis1-Hoxa9 cells at 30 days.

FIG. 9 shows expression of a repressor HFP in AML cells increases expression of Gr-1 and Mac-1 differentiation markers and decreases expression of Flt3 receptor.

FIG. 10 shows that the repressor (right handed bar in each column) elevates differentiation-specific genes (statistically significant (p<0.05); the control data is shown in the left handed column). Total RNA from the S3 transduced Hoxa9-Meis1 murine cells and vector only-transduced control cells was analyzed by QPCR for various differentiation-specific markers.

FIG. 11 shows that repressor-expressing cells induce AML with a longer latency than vector control (median survival 94 days for repressor versus 62 days for control, p<0.002). Hoxa9-Meis1 murine AML cells expressing MSCV IRES GFP vector-only or MSCV S3 IRES GFP were sorted post-transduction and 250,000 GFP positive sorted cells were transplanted into wild-type C57B1/6 mice that were sub-lethally irradiated (4.5 Gy) prior to transplantation in order to enable AML cell engraftment. Survival of the mice was determined for the two groups with n=5 mice per group.

### DETAILED DESCRIPTION OF THE INVENTION

The present invention is based, at least in part, on the discovery of cell-permeable DNA-targeting fusion proteins which act as transcription modulators. The fusion proteins provided herein comprise two homeodomain proteins and a transcription modulator domain. The transcription modulator domain can either be a transcription repressor or transcription activator domain. Exemplary homeodomains include members of the homeobox (Hox) family of proteins and PBX family of proteins. The fusion proteins are useful for the treatment of any proliferative diseases. The fusion proteins are also useful for the treatment of diseases or disorders associated with aberrant Hox activity. In certain embodiments, the fusion proteins are useful for cancer such as AML. For fusion proteins comprising a transcription repressor domain, the fusion proteins act to repress gene transcription and, therefore, in certain embodiments, enable cell differentiation in cells. For example, aberrant HoxA9 activity plays an essential role in AML progression by blocking cell differentiation. Thus, in certain embodiments, the fusion proteins comprise the HoxA9 homeodomain or variant thereof, and a transcription repressor domain capable of repressing HoxA9 transcriptional target genes. Therefore, the fusion proteins enable leukemia cell differentiation in AML patients. The inventive concepts provided herein may be applied to the >200 homeodomain proteins involved in human disease.

### Fusion Proteins

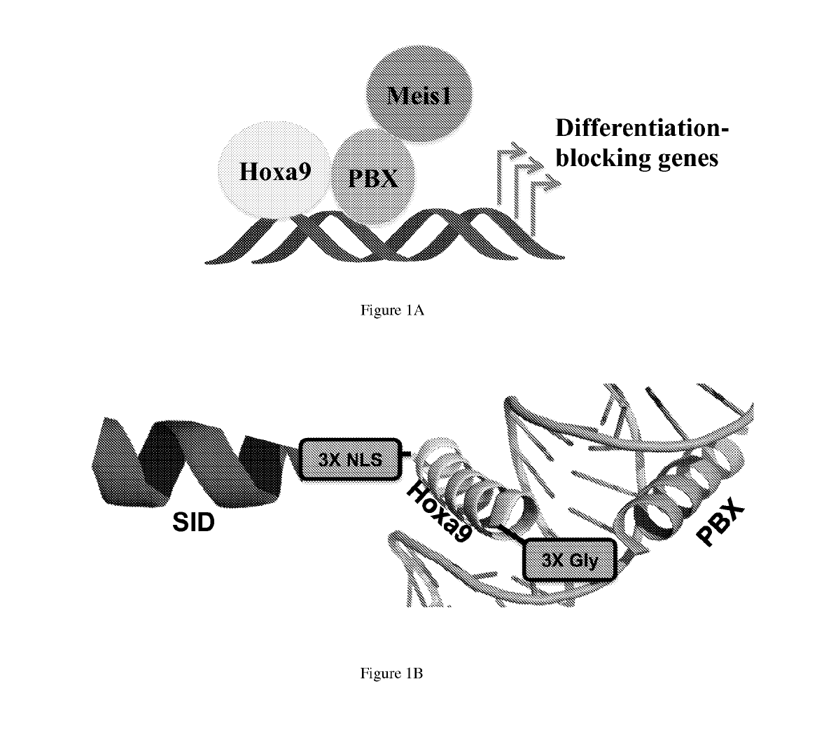

Provided herein are fusion proteins comprising a homeodomain fusion protein (HFP) domain and a transcription modulator (TM) domain. The homeodomain fusion protein domain binds to a target gene and is itself a fusion of a first homeodomain and a second homeodomain. An exemplary fusion protein is illustrated in FIG. 1B, wherein the homeodomain fusion protein domain is represented by HoxA9 and PBX and the transcription modulator domain is represented by SID. The transcription modulator domain either activates or represses transcription of a target gene. Within the HFP domain there may optionally be a non-homeodomain protein domain.

In certain embodiments, the fusion protein comprises a homeodomain fusion protein (HFP) domain, a nuclear localization sequence (NLS) domain, and a transcription modulator (TM) domain.

In certain embodiments, one of the homeodomains is alpha-helical. In certain embodiments, both the homeodomains are alpha-helical. In certain embodiments, the transcription modulator domain is alpha-helical. In certain embodiments, both the homeodomains and the TM domain are alpha-helical. In certain embodiments, either one or both of the homeodomains or transcription modulator domains is stapled or stitched.

In certain embodiments, the fusion protein comprises a HFP domain containing two full-length homeodomains. In certain embodiments, the fusion protein comprises a HFP domain containing one or two truncated homeodomains. In certain embodiments, the homeodomains are the homeodomains of Hox proteins or Pbx proteins. The fusion protein can comprise a HFP domain that can be heterodimeric or homodimeric. For example, a homodimeric HFP domain contains two of the same type of homeodomains such as a HFP domain comprising two PBX homeodomains. A heterodimeric HFP domain contains two different types of homeodomains such as a HFP domain comprising a HOX homeodomain and a PBX homeodomain.

In certain embodiments, the heterodimeric MT domain comprises a Hox homeodomain or variant thereof, and a Pbx homeodomain or variant thereof. In certain embodiments, the homodimeric homeodomain fusion protein domain comprises two Pbx homeodomains or variants thereof. In certain embodiments, the HFP domain comprises two Pbx homeodomain or variant thereof, and a E2A. protein or variant thereof.

In certain embodiments, the fusion protein binds to a DNA consensus sequence having a sequence: TGATTGAT. For example, a PBX-PBX homeodomain fusion protein binds to TGATTGAT. In certain embodiments, the fusion protein binds to a DNA consensus sequence having a sequence: TGATTNA(T/C), wherein N is T, G, A, or C. In certain embodiments, the fusion protein binds to a DNA consensus sequence having a sequence: TGATTTA(T/C), TGATTGAT, or TGATTAAT. For example, a HoxA9-Pbx1 homeodomain fusion protein binds to TGATTTAT or to TGATTTAC; a HoxA1-Pbx1 homeodomain fusion protein binds to TGATTGAT; and a HoxA5-Pbx1 homeodomain fusion protein binds to TGATTAAT. In certain embodiments, the fusion protein binds to a DNA consensus sequence having a sequence: TGATTTA(T/C). In certain embodiments, the fusion protein binds to a DNA consensus sequence having a sequence: TGATTGAT. In certain embodiments. the fusion protein binds to a DNA consensus sequence having a sequence: TGATTAAT.

In certain embodiments, the HFP domain comprises a homeodomain comprising the sequence of a full-length Hox homeodomain. For clarity, a full-length Hox homeodomain means only the homeodomain sequence of a Hox protein and does not mean the entire full-length Hox protein. In certain embodiments, a homeodomain comprises a sequence that is at least about 80% homologous to the sequence of a full-length Hox homeodomain. In certain embodiments, a homeodomain comprises a sequence that is at least about 85% homologous to the sequence of a full-length Hox homeodomain. In certain embodiments, a homeodomain comprises a sequence that is at least about 90% homologous to the sequence of a full-length Hox homeodomain. In certain embodiments, a homeodomain comprises a sequence that is at least about 95% homologous to the sequence of a full-length Hox homeodomain. In certain embodiments, a homeodomain comprises a sequence that is at least about 96% homologous to the sequence of a full-length Hox homeodomain. In certain embodiments, a homeodomain comprises a sequence that is at least about 97% homologous to the sequence of a full-length Hox homeodomain. In certain embodiments, a homeodomain comprises a sequence that is at least about 98% homologous to the sequence of a full-length Hox homeodomain. In certain embodiments, a homeodomain comprises a sequence that is at least about 99% homologous to the sequence of a full-length Hox homeodomain. The foregoing percent homology embodiments are applicable to all sequences described herein including both full-length homeodomain and truncated homeodomain sequences and to the fusion proteins provided herein.

In certain embodiments, a homeodomain comprises a sequence that is at least about 80% identical to the sequence of a full-length Hox homeodomain. In certain embodiments, a homeodomain comprises a sequence that is at least about 85% identical to the sequence of a full-length Hox homeodomain. In certain embodiments, a homeodomain comprises a sequence that is at least about 90% identical to the sequence of a full-length Hox homeodomain. In certain embodiments, a homeodomain comprises a sequence that is at least about 95% identical to the sequence of a full-length Hox homeodomain. In certain embodiments, a homeodomain comprises a sequence that is at least about 96% identical to the sequence of a full-length Hox homeodomain. In certain embodiments, a homeodomain comprises a sequence that is at least about 97% identical to the sequence of a full-length Hox homeodomain. In certain embodiments, a homeodomain comprises a sequence that is at least about 98% identical to the sequence of a full-length Hox homeodomain. In certain embodiments, a homeodomain comprises a sequence that is at least about 99% identical to the sequence of a full-length Hox homeodomain. The foregoing percent identity embodiments are applicable to all sequences described herein including both full-length homeodomain and truncated homeodomain sequences and to the fusion proteins provided herein.

In certain embodiments, the full-length Hox homeodomain is the full-length HoxA9 homeodomain of the sequence: NNPAANWLHARSTRKKRCPYTKHQTLELEKEFLFNMYLTRDRRYEVARLLNLTERQ VKIWFQNRRMKMKKINKDRAK (SEQ ID NO: 5). The foregoing homology and identity embodiments are applicable to SEQ ID NO: 5.

In certain embodiments, the HFP domain comprises a homeodomain comprising the sequence of a full-length Pbx homeodomain. For clarity, a full-length Pbx homeodomain means only the homeodomain sequence of a Pbx protein and does not mean the entire full-length Pbx protein. In certain embodiments, the homeodomain comprises the sequence of the full-length Pbx1 homeodomain. In certain embodiments, the homeodomain comprises the sequence of the full-length Pbx2 homeodomain. In certain embodiments, the homeodomain comprises the sequence of the full-length Pbx3 homeodomain. In certain embodiments, the homeodomain comprises the sequence of the full-length Pbx4 homeodomain. In certain embodiments, the sequence of a full-length Pbx homeodomain is ARRKRRNFX₉ KQATEX₁₅ LNEYFYSHLX₂₅NPYPSEEAKEELAX₃₉KX₄₁X₄₂X₄₃TX₄₅SQVS NWFGNKRIRYKKNX₆₃GKFQEEAX₇₁X₇₂ Y (SEQ ID NO: 6), wherein X₉ is N, X₁₅ is I, X₂₅ is S, X₃₉ is K, X₄₁ is C, X₄₂ is G, X₄₃ is I, X₄₅ is V, X₆₃ is I, X₇₁ is N, and X₇₂ is I; wherein X₉ is S, X₁₅ is V, X₂₅ is S, X₃₉ is K, X₄₁ is C, X₄₂ is G, X₄₃ is I, X₄₅ is V, X₆₃ is I, X₇₁ is N, and X₇₂ is I; wherein X₉ is S, X₁₅ is I, X₂₅ is S, X₃₉ is K, X₄₁ is C, X₄₂ is S, X₄₃ is I, X₄₅ is V, X₆₃ is I, X₇₁ is N, and X₇₂ is L; or wherein X₉ is S, X₁₅ is V, X₂₅ is N, X₃₉ is R, X₄₁ is G, X₄₂ is G, X₄₃ is L, X₄₅ is I, X₆₃ is M, X₇₁ is Y, and X₇₂ is I.

In certain embodiments, a homeodomain comprises a sequence that is at least about 80% homologous to the sequence of a full-length Pbx homeodomain. In certain embodiments, a homeodomain comprises a sequence that is at least about 85% homologous to the sequence of a full-length Pbx homeodomain. In certain embodiments, a homeodomain comprises a sequence that is at least about 90% homologous to the sequence of a full-length Pbx homeodomain. In certain embodiments, a homeodomain comprises a sequence that is at least about 95% homologous to the sequence of a full-length Pbx homeodomain. In certain embodiments, a homeodomain comprises a sequence that is at least about 96% homologous to the sequence of a full-length Pbx homeodomain. In certain embodiments, a homeodomain comprises a sequence that is at least about 97% homologous to the sequence of a full-length Pbx homeodomain. In certain embodiments, a homeodomain comprises a sequence that is at least about 98% homologous to the sequence of a full-length Pbx homeodomain. In certain embodiments, a homeodomain comprises a sequence that is at least about 99% homologous to the sequence of a full-length Pbx homeodomain.

In certain embodiments, a homeodomain comprises a sequence that is at least about 80% identical to the sequence of a full-length Pbx homeodomain. In certain embodiments, a homeodomain comprises a sequence that is at least about 85% identical to the sequence of a full-length Pbx homeodomain. In certain embodiments, a homeodomain comprises a sequence that is at least about 90% identical to the sequence of a full-length Pbx homeodomain. In certain embodiments, a homeodomain comprises a sequence that is at least about 95% identical to the sequence of a full-length Pbx homeodomain. In certain embodiments, a homeodomain comprises a sequence that is at least about 96% identical to the sequence of a full-length Pbx homeodomain. In certain embodiments, a homeodomain comprises a sequence that is at least about 97% identical to the sequence of a full-length Pbx homeodomain. In certain embodiments, a homeodomain comprises a sequence that is at least about 98% identical to the sequence of a full-length Pbx homeodomain. In certain embodiments, a homeodomain comprises a sequence that is at least about 99% identical to the sequence of a full-length Pbx homeodomain. In certain embodiments, the full-length Pbx homeodomain is the full-length Pbx1 homeodomain of the sequence: ARRKRRNFNKQATEILNEYFYSHLSNPYPSEEAKEELAKKCGITVSQVSNWFGNKRI RYKKNIGKFQEEANIY (SEQ ID NO: 7). The foregoing homology and identity embodiments are applicable to SEQ ID NO: 7. In certain embodiments, the full-length Pbx homeodomain is the full-length Pbx2 homeodomain of the sequence: ARRKRRNFSKQATEVLNEYFYSHLSNPYPSEEAKEELAKKCGITVSQVSNWFGNKRI RYKKNIGKFQEEANIY (SEQ ID NO: 8). The foregoing homology and identity embodiments are applicable to SEQ ID NO: 8. In certain embodiments, the full-length Pbx homeodomain is the full-length Pbx3 homeodomain of the sequence: ARRKRRNFSKQATEILNEYFYSHLSNPYPSEEAKEELAKKCSITVSQVSNWFGNKRIR YKKNIGKFQEEANLY (SEQ ID NO: 9). The foregoing homology and identity embodiments are applicable to SEQ ID NO: 9. In certain embodiments, the full-length Pbx homeodomain is the full-length Pbx4 homeodomain of the sequence: ARRKRRNFSKQATEVLNEYFYSHLNNPYPSEEAKEELARKGGLTISQVSNWFGNKRIRYKKNMGKFQEEATIY (SEQ ID NO: 10). The foregoing homology and identity embodiments are applicable to SEQ ID NO: 10.

In certain embodiments, the HFP domain comprises a first homeodomain comprising a sequence that is at least about 80% homologous to HoxA9 of SEQ ID NO: 5, and a second homeodomain comprising a sequence that is at least about 80% homologous to Pbx1 of SEQ ID NO: 6. In certain embodiments, the HFP domain comprises a first homeodomain comprising a sequence that is at least about 85%, at least about 90%, at least about 95%, at least about 96%, at least about 97%, at least about 98%, or at least about 99% homologous to HoxA9 of SEQ ID NO: 5, and a second homeodomain comprising a sequence that is at least about 85%, at least about 90%, at least about 95%, at least about 96%, at least about 97%, at least about 98%, or at least about 99% homologous to Pbx1 of SEQ ID NO: 6.

In certain embodiments, the HFP domain comprises a first homeodomain comprising a sequence that is at least about 80% identical to HoxA9 of SEQ ID NO: 5, and a second homeodomain comprising a sequence that is at least about 80% identical to Pbx1 of SEQ ID NO: 6. In certain embodiments, the HFP domain comprises a first homeodomain comprising a sequence that is at least about 85%, at least about 90%, at least about 95%, at least about 96%, at least about 97%, at least about 98%, or at least about 99% identical to HoxA9 of SEQ ID NO: 5, and a second homeodomain comprising a sequence that is at least about 85%, at least about 90%, at least about 95%, at least about 96%, at least about 97%, at least about 98%, or at least about 99% identical to Pbx1 of SEQ ID NO: 6.

In certain embodiments, the HFP domain comprises the full-length HoxA9 homeodomain of SEQ ID NO: 5 or a variant thereof, and the full-length Pbx1 homeodomain of SEQ ID NO: 6 or a variant thereof. The HFP domain comprising the HoxA9 and the Pbx1 homeodomains binds to a DNA consensus sequence having a sequence of TGATTTAT. The HoxA9 homeodomain binds to a TTA(T/C) sequence on a target gene, and the PBX homeodomain binds to a TGAT sequence on the target gene. In certain embodiments, the HFP domain comprises a homeodomain comprising the sequence of a truncated Hox homeodomain. In certain embodiments, the truncated Hox homeodomain is the truncated HoxA9 homeodomain of the sequence: X₁RQVX₅X₆WX₈X₉X₁₀RRX₁₃X₁₄X₁₅KX₁₇IN (SEQ ID NO: 1). In this sequence, X₁ is E or an amino acid capable of cross-linking with another amino acid capable of cross-linking. Each of X₅ and X₁₄ is independently any amino acid residue. Each of X₆, X₉, X₁₀, and X₁₃ is independently any amino acid residue or an amino acid capable of cross-linking with another amino acid capable of cross-linking. X₈ is F or an amino acid capable of cross-linking with another amino acid capable of cross-linking. X₁₅ is M or an amino acid capable of cross-linking with another amino acid capable of cross-linking. X₁₇ is any amino acid residue. The truncated HoxA9 homeodomain comprises at most two or three amino acids capable of cross-linking with another amino acid capable of cross-linking.

In certain embodiments, the truncated HoxA9 homeodomain comprises SEQ ID NO: 1, wherein X₅ is K, X₆ is I, X₉ is Q, X₁₀ is N, X₁₃ is M, X₁₄ is K. In certain embodiments, the truncated HoxA9 homeodomain comprises SEQ ID NO: 1, wherein X₅ is K, X₆ is I, X₉ is Q, X₁₀ is N, X₁₃ is M, X₁₄ is K, and X₁₇ is K. In certain embodiments, the truncated HoxA9 homeodomain comprises the sequence: ERQVKIWFQNRRMKMKKINK (SEQ ID NO: 2).

In certain embodiments, the HFP domain comprises a homeodomain comprising the sequence of a truncated Pbx homeodomain. In certain embodiments, the truncated Pbx homeodomain is the truncated PBX homeodomain of the sequence: X₁X₂QVSNWX₈GNKRIRX₁₅KKNIG (SEQ ID NO: 3). In the sequence, X₁ is V or an amino acid capable of cross-linking with another amino acid capable of cross-linking. X₂ is any amino acid residue. X₆ is N or an amino acid capable of cross-linking with another amino acid capable of cross-linking. X₈ is F or an amino acid capable of cross-linking with another amino acid capable of cross-linking. X₁₀ is N or an amino acid capable of cross-linking with another amino acid capable of cross-linking. X₁₄ is R or an amino acid capable of cross-linking with another amino acid capable of cross-linking. X₁₅ is Y or an amino acid capable of cross-linking with another amino acid capable of cross-linking. The truncated Pbx homeodomain comprises at most two or three amino acids capable of cross-linking with another amino acid capable of cross-linking. In certain embodiments, the truncated Pbx homeodomain comprises SEQ ID NO: 3, wherein X₂ is S. In certain embodiments, the truncated PBX domain comprises a sequence: VSQVSNWFGNKRIRYKKNIG (SEQ ID NO: 4). Pbx1, Pbx2, and Pbx3 have the same truncated PBX homeodomain sequence.

The nuclear localization sequence (NLS) domain comprises a NLS. In certain embodiments, the NLS is SV40 NLS. In certain embodiments, the NLS is DPKKKRKV (SEQ ID NO: 18). In certain embodiments, the NLS is PKKKRKV (SEQ ID NO: 19). The NLS can be is repeated two or three times within the NLS domain. In certain embodiments, the NLS domain comprises the sequence: DPKKKRKVDPKKKRKV (SEQ ID NO: 20). In certain embodiments, the NLS domain comprises the sequence: PKKKRKVPKKKRKV (SEQ ID NO: 21). In certain embodiments, the NLS domain comprises the sequence: DPKKKRKVDPKKKRKVDPKKKRKV (SEQ ID NO: 22). In certain embodiments, the NLS domain comprises the sequence: PKKKRKVPKKKRKVPKKKRKV (SEQ ID NO: 23).

The arrangement of the various domains of the HFP domain can be varied. In certain embodiments, the NLS domain is located at the N-terminal side of the HFP domain. In certain embodiments, the NLS domain is located at the C-terminal side of the HFP domain. In certain embodiments, the NLS is located between the HFP domain and the TM domain. Provided below is a schematic of the exemplary ways in which the various domains can be arranged:

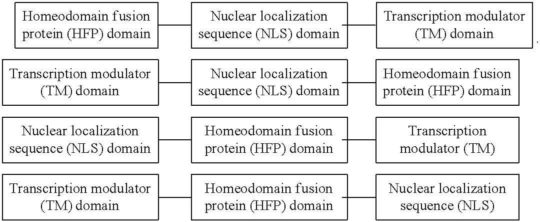

The top scheme illustrates how the HFP domain is located at the N-terminal side relative to the TM domain with the NLS domain between the HFP and TM domains. The second scheme illustrates how the HFP domain is located at the C-terminal side relative to the TM domain with the NLS domain between the HFP and TM domains. The third scheme illustrates how the NLS is located at the N-terminal side relative to the HFP domain and the TM domain. The fourth scheme illustrates how the NLS is located at the N-terminal side relative to the HFP domain and the TM domain. Other additional arrangements may be possible. For example, the NLS domain may be located within the HFP domain between the first and the second homeodomains.

Within the HFP domain, the two homeodomains can be arranged in either a forward or reverse assembly. In certain embodiments, the first homeodomain is located at the N-terminal end, and the second homeodomain is located at the C-terminal end (forward assembly). In certain embodiments, the first homeodomain is located at the C-terminal end, and the second homeodomain is located at the N-terminal end (reverse assembly). Provided below is a schematic of the exemplary ways of how the homeodomains can be arranged:

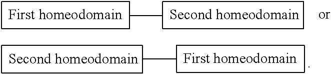

The top scheme illustrates how the first homeodomain is located at the N-terminal side relative to the second homeodomain. The bottom scheme illustrates how the first homeodomain is located at the C-terminal side relative to the second homeodomain. For example, if the first homeodomain is Hox homeodomain and the second homeodomain is Pbx homeodomain, then in the top scheme, Hox homeodomain would be at the N-terminal end relative to the Pbx homeodomain. In the second scheme, Hox homeodomain would be at the C-terminal end relative to the Pbx homeodomain.

Various linkers are known in the art for joining peptides and proteins. It will be appreciated that the length of the linker L is variable and can be designed based on the required flexibility or rigidity necessary to link the peptides. In certain embodiments, the linker is a bond or a polymer with optional functional groups. The functional group could be one or more atoms, for example, an amide, ester, ether, or disulfide. The polymer can be natural or unnatural. The polymer can be a peptide. This linker can have any length or other characteristic and minimally comprises two reactive terminal groups that can chemically interact with (and covalently bind to) the peptides or proteins. some embodiments, the linker can comprise natural or non-natural amino acids and/or may comprise other molecules with terminal reactive groups. For example, NHS or maleimide reactive terminal groups, such as, SM(PEG)ₙ Succinimidyl-([N-maleimidopropionamido]-n-ethyleneglycol). Other linkers that can be used to join the homeodomains include polyethylene glycol (PEG) linkers or polyglycine (glycine-repeats) linkers.

In certain embodiments, the first homeodomain and the second homeodomain are fused together using a polyglycine linker. In certain embodiments, the first truncated homeodomain and the second truncated homeodomain are fused together using a linker that is one glycine long (-G-). In certain embodiments, the linker is two glycines long (-GG-). In certain embodiments, the linker is three glycines long (-GGG-). In certain embodiments, the linker is four glycines long (-GGGG-)(SEQ ID NO: 55). In certain embodiments, the linker is five glycines long (-GGGGG-)(SEQ ID NO: 56). Other amino acids can be found with a polyglycine linker such as serine. In certain embodiments, the first full-length homeodomain and the second full-length homeodomain are fused together using a long flexible linker. In certain embodiments, the linker is (-SGGGGS-)ₙ (SEQ ID NO: 57) wherein n is 1 to 4. In certain embodiments, the linker is -SGGGGS- (SEQ ID NO: 57). In certain embodiments, the long flexible linker is -SGGGGSGGGGS- (SEQ ID NO: 58).

In certain embodiments, the linker is 1-5 amino acids long, 5-10 amino acids long, 1-10 amino acids long, 10-15 amino acids long, 15-20 amino acids long, 1-20 amino acids long, 25-30 amino acids long, or 1-30 amino acids long.

The fusion protein have have various lengths or molecular weights. In certain embodiments, the fusion protein comprising the homeodomain fusion protein domain, a nuclear localization sequence (NLS) domain, and the transcription modulator domain, is less than about 180 amino acids long. In certain embodiments, the fusion protein is less than about 170 amino acids long. In certain embodiments, the fusion protein is less than about 160 amino acids long. In certain embodiments, the fusion protein is less than about 150 amino acids long. In certain embodiments, the fusion protein is less than about 140 amino acids long. In certain embodiments, the fusion protein is less than about 130 amino acids long. In certain embodiments, the fusion protein is less than about 120 amino acids long. In certain embodiments, the fusion protein is less than about 110 amino acids long. In certain embodiments, the fusion protein is less than about 100 amino acids long. In certain embodiments, the fusion protein is less than about 90 amino acids long. In certain embodiments, the fusion protein is less than about 80 amino acids long. In certain embodiments, the fusion protein is less than about 70 amino acids long. In certain embodiments, the fusion protein is less than about 60 amino acids long.

In certain embodiments, the fusion protein comprising the homeodomain fusion protein domain, a nuclear localization sequence (NLS) domain, and the transcription modulator domain has a molecular weight range of about 8,500 to about 20,000 Da. In certain embodiments, the fusion protein has a molecular weight range of about 8,800 to about 12,700 Da. In certain embodiments, the fusion protein has a molecular weight range of about 8,500 to about 10,000 Da. In certain embodiments, the fusion protein has a molecular weight range of about 8,500 to about 15,000 Da. In certain embodiments, the fusion protein has a molecular weight range of about 8,500 to about 13,000 Da. In certain embodiments, the fusion protein has a molecular weight range of about 10,000 to about 15,000 Da. In certain embodiments, the fusion protein has a molecular weight range of about 15,000 to about 20,000 Da.

In certain embodiments, the fusion protein has a molecular weight range of at most about 15,000 Da. In certain embodiments, the fusion protein has a molecular weight range of at most about 12,500 Da. In certain embodiments, the fusion protein has a molecular weight range of at most about 12,000 Da. In certain embodiments, the fusion protein has a molecular weight range of at most about 11,500 Da. In certain embodiments, the fusion protein has a molecular weight range of at most about 11,000 Da. In certain embodiments, the fusion protein has a molecular weight range of at most about 10,500 Da. In certain embodiments, the fusion protein has a molecular weight range of at most about 10,000 Da. In certain embodiments, the fusion protein has a molecular weight range of at most about 9,500 Da. In certain embodiments, the fusion protein has a molecular weight range of at most about 9,000 Da.

In certain embodiments, the fusion protein comprises a transcription modulator (TM) domain that is about 15-20 amino acids long. In certain embodiments, TM domain is about 20-25 amino acids long. In certain embodiments, TM domain is about 25-30 amino acids long. In certain embodiments, TM domain is about 30-35 amino acids long. In certain embodiments, TM domain is about 35-40 amino acids long. In certain embodiments, TM domain is about 40-45 amino acids long. In certain embodiments, the fusion protein comprises a homeodomain fusion protein domain, and a transcription modulator domain that is a transcription repressor domain with any of the foregoing lengths. In certain embodiments, the fusion protein comprises a homeodomain fusion protein domain, and a transcription modulator domain that is a transcription activator domain with any of the foregoing lengths.

In certain embodiments, the transcription repressor (TR) domain is at most 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 amino acids long. In certain embodiments, the TR domain is at most 15 amino acids long. In certain embodiments, the TR domain is at most 20 amino acids long. In certain embodiments, the TR domain is at most 25 amino acids long. In certain embodiments, the TR domain is at most 30 amino acids long. In certain embodiments, the TR domain is at most 35 amino acids long. In certain embodiments, the TR domain is at most 40 amino acids long. In certain embodiments, the TR domain is at most 45 amino acids long. In certain embodiments, the fusion protein comprises a transcription modulator domain that is a transcription activator domain with any of the foregoing lengths.

Non-limiting examples of TR domains include sin3-interacting domain (SID) and Krüppel associated box (KRAB) domain sequences. In certain embodiments, the TR domain comprises a SID sequence or variant thereof. In certain embodiments, the TR domain is a SID variant selected from MATAVGMNIQLLLEAADYLERREREAEHGYASMLPY (SEQ ID NO: 11), MVGMNIQLLLEAADYLERREREAEH (SEQ ID NO: 12), MVGMNIQLLLEAADYLERRER (SEQ ID NO: 13), MVGMNIQLLLEAADYLE (SEQ ID NO: 14), MNIQLLLEAADYLERRER (SEQ ID NO: 15), MNIQLLLEAADYLE (SEQ ID NO: 16), NIQLLLEAADYLER (SEQ ID NO: 17). The first methionine in SEQ ID NO: 11 is optional and not required for SID activity. In certain embodiments, the TR domain comprises a SID sequence that is at least about 80%, 83%, 85%, 87%, 90%, 95%, 96%, 97% homologous to the SID sequences of SEQ ID NO: 11-17.

In certain embodiments, the TR domain comprises a KRAB domain sequence. In certain embodiments, the KRAB sequence comprises the sequence: RTLVTFKDVFVDFTREEWKLLDTAQQIVYRNVMLENYKNLVSL (SEQ ID NO: 24). In certain embodiments, the TR domain comprises a KRAB domain sequence that is at least about 80%, 83%, 85%, 87%, 90%, 95%, 96%, 97% homologous to the KRAB sequence of SEQ ID NO: 24.

Non-limiting examples of transcription activator domains include VP16, VP64 (4XVP16)TAF9, CBP/p300, and CBP/p300 domains which include TAZ1, TAZ2, NCBD, and KIX. In certain embodiments, the transcription activator domain comprises a VP16 sequence. In certain embodiments, the transcription activator domain comprises a VP32 sequence. In certain embodiments, the transcription activator domain comprises a VP48 sequence. In certain embodiments, the transcription activator domain comprises a VP64 sequence. The VP32, VP48, and VP64 sequences are a VP16 sequence repeated two, three, and four times, respectively (2XVP16, 3X VP16, 4XVP16). In certain embodiments, the transcription activator domain comprises a KIX CBP sequence.

In certain embodiments, the fusion protein comprises at least one homeodomain comprising an alpha-helix nucleating motif sequence. In certain embodiments, the alpha-helix nucleating motif sequence is located at the N-terminus of a homeodomain. In certain embodiments, the alpha-helix nucleating motif sequence is located at the N-terminus of a TM domain. In certain embodiments, the alpha-helix nucleating motif sequence is the amino acid residues of DP, NP, DPA, or NPA. In certain embodiments, the alpha-helix nucleating motif sequence is DP or NP. In certain embodiments, the alpha-helix nucleating motif sequence is DP. In certain embodiments, an additional A is included in the alpha-helix nucleating motif sequence to enable greater helical stabilization. In certain embodiments, the alpha-helix nucleating motif sequence is DPA or NPA. In certain embodiments, the alpha-helix nucleating motif sequence is DPA.

In certain embodiments, the fusion protein comprises a homeodomain fusion protein (HFP) domain, a nuclear localization sequence (NLS) domain, and a transcription modulator (TM) domain, wherein the homeodomain fusion protein domain comprises a first homeodomain comprising a HoxA9 sequence of SEQ ID NO: 2, and a second homeodomain comprising a Pbx1 sequence of SEQ ID NO: 4, and the transcription modulator domain is a transcription repressor (TR) domain. In certain embodiments, the fusion protein binds to the DNA consensus sequence having a sequence of TGATTTAT. In certain embodiments, at least one of or both of the Hoxa9 and Pbx1 sequences comprises an alpha-helix nucleating motif of DP or NP. In certain embodiments, both of the Hoxa9 and Pbx1 homeodomain sequences comprises an alpha-helix nucleating motif of DP. In certain embodiments, the Hoxa9 and Pbx1 sequences are connected using a polyglycine linker. In certain embodiments, the polyglycine linker is three glycines long. In certain embodiments, the TR domain is a sin3-interacting domain (SID) selected from SEQ ID NO: 11 to 17. In certain embodiments, the TM domain comprising SID is located on the N-terminal side of the fusion protein, and the HFP domain is located on the C-terminal side of the fusion protein. In certain embodiments, the TM domain comprising SID is located on the C-terminal side of the fusion protein, and the HFP domain is located on the N-terminal side of the fusion protein. In certain embodiments, the TM domain comprises an alpha-helix nucleating motif sequence that is DPA or NPA. In certain embodiments, the alpha-helix nucleating motif sequence is located at the N-terminal side of the TM domain. In certain embodiments, the fusion protein comprises an NLS domain comprising SEQ ID NO: 18, 19, 20, 21, 22 or 23. In certain embodiments, the fusion protein comprises an NLS domain comprising SEQ ID NO: 22. In certain embodiments, either one or both of the homeodomains and transcription modulator domains are stapled or stitched.

In certain embodiments, the fusion protein comprises a homeodomain fusion protein (HFP) domain, a nuclear localization sequence (NLS) domain, and a transcription modulator (TM) domain, wherein the homeodomain fusion protein domain comprises first homeodomain comprising a HoxA9 sequence of SEQ ID NO: 2, and a second homeodomain comprising a Pbx1 sequence of SEQ ID NO: 4; and the transcription modulator domain is a transcription repressor (TR) domain comprising a sin3-interacting domain (SID) having a sequence: MATAVGMNIQLLLEAADYLERREREAEHGYASMLPY (SEQ ID NO: 11). In certain alternative embodiments, the sin3-interacting domain (SID) has the sequence: MVGMNIQLLLEAADYLERREREAEH (SEQ ID NO: 12). In certain alternative embodiments, the sin3-interacting domain (SID) has the sequence: MVGMNIQLLLEAADYLERRER (SEQ ID NO: 13).

The DNA binding specificity of the fusion proteins can be engineered by randomizing amino acids in one or both homeodomains of the homeodomain fusion protein domain. For example, a HFP domain comprising a HoxA9 sequence of SEQ ID NO: 2 and a Pbx1 sequence of SEQ ID NO: 4 can be engineered by randomizing at least one amino acid in one homeodomain while keeping the amino acids in the other homeodomain unmutated. For example, SEQ ID NO: 4 can be kept unchanged to serve as an anchor for the TGAT binding site, and various positions of SEQ ID NO: 2 such as the amino acids at positions 5, 6, 9, 10, 13, or 14 can be randomized to create a screening library for targeting a desired DNA sequence. An exemplary DNA that may be used for screening comprises TGATNNNN, wherein each N is independently T, G, A, or C.

The fusion proteins provided herein are prepared to be cell-permeable. In certain embodiments, the fusion protein comprises an anthrax toxin lethal factor. In certain embodiments, the anthrax toxin lethal factor is the N-terminal portion of anthrax toxin lethal factor (LF_{N}). In certain embodiments, the anthrax toxin lethal factor is located at the N-terminal end of the fusion protein. In certain embodiments, the fusion protein comprises at least one additional NLS at the N-terminal end of the anthrax toxin lethal factor. In certain embodiments, the fusion protein comprises at least one additional NLS embedded into the sequence of the N-terminal end of the anthrax toxin lethal factor. In certain embodiments, the fusion protein comprises an anthrax toxin lethal factor. In certain embodiments, the anthrax toxin lethal factor is the N-terminal portion of anthrax toxin lethal factor (LF_{N}) comprising the sequence:

(SEQ ID NO: 25)

MGSSHHHHHHSSGLVPRGSHMAGGHGDVGMHVKEKEKNKDENKRKDEERN
KTQEEHLKEIMKHIVKIEVKGEEAVKKEAAEKLLEKVPSDVLEMYKAIGG
KIYIVDGDITKHISLEALSEDKKKIKDIYGKDALLHEHYVYAKEGYEPVL
VIQSSEDYVENTEKALNVYYEIGKILSRDILSKINQPYQKFLDVLNTIKN
ASDSDGQDLLFTNQLKEHPTDFSVEFLEQNSNEVQEVFAKAFAYYIEPQH
RDVLQLYAPEAFNYMDKFNEQEINLSLEELKDQRSGRELE.

In certain embodiments, the fusion protein comprising an anthrax toxin lethal factor has a molecular weight range of about 45,000 Da to about 55,000 Da. In certain embodiments, the fusion protein comprising an anthrax toxin lethal factor has a molecular weight range of about 45,000 Da to about 50,000 Da. In certain embodiments, the fusion protein comprising an anthrax toxin lethal factor has a molecular weight range of about 50,000 Da to about 55,000 Da.

In certain embodiments, the fusion protein comprising an anthrax toxin lethal factor has a molecular weight range of at most about 55,000 Da. In certain embodiments, the fusion protein comprising an anthrax toxin lethal factor has a molecular weight range of at most about 50,000 Da.

The fusion proteins provided herein are capable of modulating the transcription of any target gene. In certain embodiments, the fusion proteins provided herein are capable of modulating the transcription of any target gene of a homeodomain protein. In certain embodiments, the fusion proteins provided herein are capable of modulating the transcription of any target gene of Hox. In certain embodiments, the fusion proteins provided herein are capable of modulating the transcription of any target gene of Pbx. In certain embodiments, a target gene is SOX4, CD34, FLT3R, FOXP1, or DNAJC10. The fusion proteins are capable of repressing the transcription of a target gene. The fusion proteins cause cell differentiation, for example, in AML cells. Thus, the fusion protein upregulates one or more differentiation-specific genes including but not limited to S 100A8, myeloperoxidase, or neutrophil elastase. The fusion protein results in an increase in expression of myeloid differentiation markers such as Mac-1 or Gr-1.

### Polynucleotides, Vectors, Cells

Provided herein are polynucleotides encoding any inventive fusion protein provided herein. For example, provided herein are polynucleotides encoding a fusion protein comprising a homeodomain fusion protein (HFP) domain, a nuclear localization sequence (NLS) domain, and a transcription modulator (TM) domain, wherein the homeodomain fusion protein domain comprises first homeodomain comprising a HoxA9 sequence of SEQ ID NO: 2 and a second homeodomain comprising a Pbx1 sequence of SEQ ID NO: 4, and the transcription modulator domain is a transcription repressor (TR) domain.

Provided herein are also nucleic acid constructs comprising the inventive polynucleotides. In addition, provided herein are expression vectors (“vectors”) comprising the inventive polynucleotides.

The term “vector” refers to a carrier DNA molecule into which a nucleic acid sequence can be inserted for introduction into a host cell. Vectors useful in the methods provided may include additional sequences including, but not limited to one or more signal sequences and/or promoter sequences, or a combination thereof. An “expression vector” is a specialized vector that contains the necessary regulatory regions needed for expression of a gene of interest in a host cell such as transcription control elements (e.g. promoters, enhancers, and termination elements). Expression vectors and methods of their use are well known in the art. Non-limiting examples of suitable expression vectors and methods for their use are provided herein.

Provided herein are cells comprising an inventive fusion protein, an inventive vector, or a fusion protein as described herein. Cells that are useful according to the invention include eukaryotic and prokaryotic cells. Eukaryotic cells include cells of non-mammalian invertebrates, such as yeast, plants, and nematodes, as well as non-mammalian vertebrates, such as fish and birds. The cells also include mammalian cells, including human cells. The cells also include immortalized cell lines such as HEK, HeLa, CHO, 3T3, which may be particularly useful in applications of the methods for drug screens. The cells also include stem cells, pluripotent cells, progenotir cells, and induced pluripotent cells. Differentiated cells including cells differentiated from the stem cells, pluripotent cells and progenitor cells are included as well. In certain embodiments, the cells are hematopoietic stem cells (HSC). In some embodiments, the cells are cultured in vitro or ex vivo. In some embodiments, the cells are part of an organ or an organism.

### Composition and Pharmaceutical Compositions

Provided herein are compositions comprising a pore-forming toxin unit and an inventive fusion protein provided herein. The compositions provided are useful for delivering the inventive fusion proteins into cells. A pore-forming toxin is prepared from a microbial toxin or modified microbial toxins.

Modified microbial toxin receptors for delivering of agents into cells have been discuss, for example, in PCT publication, WO 2013/126690, and US application , U.S. Ser. No. 61/602,218, the entire contents of which are incorporated herein by reference. The anthrax toxin (ATx) is an ensemble of three large proteins: Protective Antigen (PA, 83 kDa), Lethal Factor (LF, 90 kDa), and Edema Factor (EF, 89 kDa). LF and EF are intracellular effector proteins: enzymes that modify substrates residing within the cytosolic compartment of mammalian cells. LF is a metalloprotease that cleaves most members of the MAP kinase family, and EF is a calmodulin- and Ca²⁺-dependent adenylyl cyclase, which elevates the level of cAMP within the cell. PA, the third component of the ensemble, is a receptor-binding transporter capable of forming pores in the endosomal membrane. These pores mediate the translocation of EF, LF, or various fusion proteins containing the N-terminal PA-binding domain of EF or LF, across the endosomal membrane to the cytosol.

Anthrax toxin uses a homopolymeric pore structure formed by protective antigen (PA) for the delivery of two alternative moieties, edema factor (EF) and lethal factor (LF), into the cytoplasm. The receptor-targeted PA variants of the present embodiments can deliver a wide variety of therapeutic proteins, both nontoxic and toxic, to chosen class or classes of cells including the toxic native A-moieties (EF and LF). For example, an inventive fustion protein is fused to the N-terminal portion of the lethal factor of anthrax toxin (LFN), and undergoes translocation through the PA variant to the target cell cytosol.

ATx action at the cellular level is initiated when PA binds to either of two receptors, ANTXR1 and ANTXR2, and is activated by a furin-class protease. The cleavage yields a 20-kDa fragment, PA20, which is released into the surrounding medium, and a 63-kDa fragment, PA63, which remains bound to the receptor. Receptor-bound PA63 spontaneously self-associates to form ring-shaped heptameric and octameric oligomers (prepores), which are capable of binding LF and/or EF with nanomolar affinity. The resulting heterooligomeric complexes are endocytosed and delivered to the endosomal compartment, where the acidic pH induces the prepores to undergo a major conformational rearrangement that allows them to form pores in the endosomal membrane.

Provided herein are compositions comprising a pore-forming toxin unit and an inventive fusion protein provided herein. The compositions provided are useful for delivering the inventive fusion proteins into cells.

In certain embodiments, the pore-forming toxin unit is a protective antigen. In certain embodiments, the fusion protein comprises a complementary toxin domain. The pore-forming toxin unit associates with the complentary toxin domain of the fusion protein. In certain embodiments, the complementary toxin domain is LF_{N} of SEQ ID NO: 25. In certain embodiments, the protective antigen associates with a LF_{N} of SEQ ID NO: 25. In certain embodiments, the protective-antigen is wild-type protective-antigen of sequence:

(SEQ ID NO: 26) [symbol] [symbol] [symbol] [symbol]
EVKQENRLLNESE
SSSQGLLGYYFSDLNFQAPMVVTSSTTGDLSIPSSELENIPSENQYFQS
AIWSGFIKVKKSDEYTFATSADNHVTMWVDDQEVINKASNSNKIRLEKG
RLYQIKIQYQRENPTEKGLDFKLYWTDSQNKKEVISSDNLQLPELKQKS
SNSRKKRSTSAGPTVPDRDNDGIPDSLEVEGYTVDVKNKRTFLSPWISN
IHEKKGLTKYKSSPEKWSTASDPYSDFEKVTGRIDKNVSPEARHPLVAA
YPIVHVDMENIILSKNEDQSTQNTDSQTRTISKNTSTSRTHTSEVHGNA
EVHASFFDIGGSVSAGFSNSNSSTVAIDHSLSLAGERTWAETMGLNTAD
TARLNANIRYVNTGTAPIYNVLPTTSLVLGKNQTLATIKAKENQLSQIL
APNNYYPSKNLAPIALNAQDDFSSTPITMNYNQFLELEKTKQLRLDTDQ
VYGNIATYNFENGRVRVDTGSNWSEVLPQIQETTARIIFNGKDLNLVER
RIAAVNPSDPLETTKPDMTLKEALKIAFGFNEPNGNLQYQGKDITEFDF
NFDQQTSQNIKNQLAELNATNIYTVLDKIKLNAKMNILIRDKRFHYDRN
NIAVGADESVVKEAHREVINSSTEGLLLNIDKDIRKILSGYIVEIEDTE
GLKEVINDRYDMLNISSLRQDGKTFIDFKKYNDKLPLYISNPNYKVNVY
AVTKENTIINPSENGDTSTNGIKKILIFSKKGYEIG.

Anthrax Protective antigen, with a 29 amino acid signal peptide marked with bold and italized; UniProtKB NO. P13423 (PAG_BACAN)

A mutant of protective-antigen has been described in Mechaly, et al. (2012) Changing the Receptor Specificity of Anthrax Toxin. mBio. 3(3): e00088-12 (available at: mbio.asm.Org/content/3/3/e00088-12) and Mccluskey, et al. (2012) Targeting HER2-positive cancer cells with receptor-redirected anthrax protective antigen. Molecular Oncology. 7(3): 440-451, each of which are entirely incorporated herein by reference. In certain embodiments, the protective-antigen is mutant protective-antigen. In certain embodiments, the mutant protective-antigen comprises mutations N682A and D683A. In certain embodiments, the mutant protective-antigen comprises the sequence:

(SEQ ID NO: 27)

EVKQENRLLNE SESSSQGLLG
YYFSDLNFQA PMVVTSSTTG DLSIPSSELENIPSENQYFQ
SAIWSGFIKV KKSDEYTFAT SADNHVTMWV DDQEVINKAS
NSNKIRLEKG RLYQIKIQYQ RENPTEKGLD FKLYWTDSQN
KKEVISSDNL QLPELKQKSS NSRKKRSTSA GPTVPDRDND
GIPDSLEVEG YTVDVKNKRT FLSPWISNIH
EKKGLTKYKS SPEKWSTASD PYSDFEKVTG RIDKNVSPEA
RHPLVAAYPI VHVDMENIIL SKNEDQSTQN TDSQTRTISK
NTSTSRTHTS EVHGNAEVHA SFFDIGGSVS AGFSNSNSST
VAIDHSLSLA GERTWAETMG LNTADTARLN
ANIRYVNTGT APIYNVLPTT SLVLGKNQTL ATIKAKENQL
SQILAPNNYY PSKNLAPIAL NAQDDFSSTP rfMNYNQFLE
LEKTKQLRLD TDQVYGNIAT YNFENGRVRV DTGSNWSEVL
PQIQETTARI IFNGKDLNLV ERRIAAVNPS
DPLETTKPDM TLKEALKIAF GFNEPNGNLQ YQGKDITEFD
FNFDQQTSQN IKNQLAELNA TNIYTVLDKI KLNAKMNILI
RDKRFHYDRN NIAVGADESV VKEAHREVIN SSTEGLLLNI
DKDIRKILSG YIVEIEDTEG LKEVINDRYD
MLNISSLRQD GKTFIDFKKYNDKLPLYISN PNYKVNVYAV
TKENTIINPS ENGDTSTNGI KKILIFSKKG YEIG;

N682 and D683 are underlined and bolded.

In certain embodiments, the mutant protective-antigen is fused to a cell-targeting domain. In certain embodiments, the cell-targeting domain is an antibody. In certain embodiments, the cell-targeting domain is an antibody that is a single-chain variable fragment (scFv). In certain embodiments, the cell-targeting domain is linked to the C-terminus of the mutant protective-antigen. In certain embodiments, the cell-targeting domain is linked to the N-terminus of the mutant protective-antigen.

Antibodies specific for various cell markers can be utilized in the methods and compositions provided herein. Various leukemia markers include, but are not limited to, CD45 (pan-hematopoietic marker), CD33, Mac-1 (CD11b), Flt3R (CD135), c-kit (CD117), or CD34. CD33 is expressed in about 80% of AML blasts. In certain embodiments, the antibody specifically binds CD33. In certain embodiments, the antibody specifically binds CD45. In certain embodiments, the antibody is a single-chain variable fragment (scFv). In certain embodiments, the antibody is a single-chain variable fragment that specifically binds CD33. In certain embodiments, the antibody is a single-chain variable fragment that specifically binds CD45. Other antibodies targeting additional cellular markers are known in the art and can be useful for the inventions described herein.

### Pharmaceutical Compositions

Provided herein are pharmaceutical compositions comprising the fursion protein compositions as described herein and a pharmaceutically acceptable excipient or carrier. Pharmaceutical compositions are for therapeutic use. Such compositions may optionally comprise one or more additional therapeutically active agents. In accordance with some embodiments, a method of administering a pharmaceutical composition comprising an inventive composition to a subject in need thereof is provided. In some embodiments, the inventive composition is administered to humans. For the purposes of the present disclosure, the “active ingredient” generally refers to fusion proteins as described herein.

Although the descriptions of pharmaceutical compositions provided herein are principally directed to pharmaceutical compositions for administration to humans, it will be understood by the skilled artisan that such compositions are generally suitable for administration to animals of all sorts. Modification of pharmaceutical compositions for administration to various animals is well understood, and the ordinarily skilled veterinary pharmacologist can design and/or perform such modification with merely ordinary, if any, experimentation.

The formulations of the pharmaceutical compositions described herein may be prepared by any method known or hereafter developed in the art of pharmacology. In general, such preparatory methods include the step of bringing the active ingredient into association with a carrier and/or one or more other accessory ingredients, and then, if necessary and/or desirable, shaping and/or packaging the product into a desired single- or multi-dose unit.

As used herein, the phrase “pharmaceutically acceptable” refers to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable excipient will not promote the raising of an immune response to an agent with which it is admixed, unless so desired. The preparation of a pharmacological composition that contains active ingredients dissolved or dispersed therein is well understood in the art and need not be limited based on formulation.

Pharmaceutical compositions may comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro, (Lippincott, Williams & Wilkins, Baltimore, Md., 2006) discloses various carriers used in formulating pharmaceutical compositions and known techniques for the preparation thereof. Except insofar as any conventional carrier medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, the pharmaceutically acceptable excipient is at least 95%, 96%, 97%, 98%, 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In some embodiments, the preservative is an anti-oxidant. In other embodiments, the preservative is a chelating agent.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Typically the pharmaceutical compositions are prepared as injectable either as liquid solutions or suspensions, however, solid forms suitable for solution, or suspensions, in liquid prior to use can also be prepared. The preparation can also be emulsified or presented as a liposome composition.

Liquid dosage forms for oral and parenteral administration include, but are not limited to, pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In some embodiments for parenteral administration, the polypeptides of the disclosure are mixed with solubilizing agents such as Cremophor, alcohols, oils, modified oils, glycols, polysorbates, cyclodextrins, polymers, and combinations thereof.

Injectable preparations, for example, sterile injectable aqueous or oleaginous suspensions may be formulated according to the known art using suitable dispersing or wetting agents and suspending agents. The sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polethylene glycols and the like.

The active ingredients can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient may be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

General considerations in the formulation and/or manufacture of pharmaceutical agents may be found, for example, in Remington: The Science and Practice of Pharmacy 21^{st} ed., Lippincott Williams & Wilkins, 2005.

Inventive fusion proteins provided herein are typically formulated in dosage unit form for ease of administration and uniformity of dosage. It will be understood, however, that the total daily usage of the compositions of the present invention will be decided by the attending physician within the scope of sound medical judgment. The specific therapeutically effective dose level for any particular subject will depend upon a variety of factors including the disease, disorder, or disorder being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The fusion proteins provided herein or pharmaceutical composition thereof, may be administered by any suitable route. In some embodiments, the peptide or pharmaceutical composition thereof, are administered by a variety of routes, including oral and intravenous. Specifically contemplated routes are systemic intravenous injection, regional administration via blood and/or lymph supply, and/or direct administration to an affected site. In general the most appropriate route of administration will depend upon a variety of factors including the nature of the agent (e.g., its stability in the environment of the gastrointestinal tract), and the disorder of the subject (e.g., whether the subject is able to tolerate oral administration). The invention encompasses the delivery of the inventive pharmaceutical composition by any appropriate route taking into consideration likely advances in the sciences of drug delivery.

In certain embodiments, the fusion proteins or pharmaceutical composition thereof, may be administered at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. The exact amount of an inventive peptide required to achieve an effective amount will vary from subject to subject, depending, for example, on species, age, and general disorder of a subject, severity of the side effects or disorder, identity of the particular compound(s), mode of administration, and the like.

In some embodiments, the present invention encompasses “therapeutic cocktails” comprising inventive fusion proteins. It will be appreciated that inventive fusion proteins and pharmaceutical compositions of the present invention can be employed in combination therapies. The particular combination of therapies (therapeutics or procedures) to employ in a combination regimen will take into account compatibility of the desired therapeutics and/or procedures and the desired therapeutic effect to be achieved. It will be appreciated that the therapies employed may achieve a desired effect for the same purpose (for example, an inventive conjugate useful for detecting tumors may be administered concurrently with another agent useful for detecting tumors), or they may achieve different effects (e.g., control of any adverse effects).

Pharmaceutical compositions of the present invention may be administered either alone or in combination with one or more therapeutically active agents. By “in combination with,” it is not intended to imply that the agents must be administered at the same time and/or formulated for delivery together, although these methods of delivery are within the scope of the invention. The compositions can be administered concurrently with, prior to, or subsequent to, one or more other desired therapeutics or medical procedures. In general, each agent will be administered at a dose and/or on a time schedule determined for that agent. Additionally, the invention encompasses the delivery of the inventive pharmaceutical compositions in combination with agents that may improve their bioavailability, reduce and/or modify their metabolism, inhibit their excretion, and/or modify their distribution within the body. It will further be appreciated that therapeutically active agent and the inventive peptides utilized in this combination may be administered together in a single composition or administered separately in different compositions.

The particular combination employed in a combination regimen will take into account compatibility of the therapeutically active agent and/or procedures with the inventive fusion protein and/or the desired therapeutic effect to be achieved. It will be appreciated that the combination employed may achieve a desired effect for the same disorder (for example, an inventive peptide may be administered concurrently with another therapeutically active agent used to treat the same disorder), and/or they may achieve different effects (e.g., control of any adverse effects).

As used herein, a “therapeutically active agent” refers to any substance used as a medicine for treatment, prevention, delay, reduction or amelioration of a disorder, and refers to a substance that is useful for therapy, including prophylactic and therapeutic treatment. A therapeutically active agent also includes a compound that increases the effect or effectiveness of another compound, for example, by enhancing potency or reducing adverse effects of the inventive peptides.

In certain embodiments, a therapeutically active agent is an anti-cancer agent, antibiotic, anti-viral agent, anti-HIV agent, anti-parasite agent, anti-protozoal agent, anesthetic, anticoagulant, inhibitor of an enzyme, steroidal agent, steroidal or non-steroidal anti-inflammatory agent, antihistamine, immunosuppressant agent, anti-neoplastic agent, antigen, vaccine, antibody, decongestant, sedative, opioid, analgesic, anti-pyretic, birth control agent, hormone, prostaglandin, progestational agent, anti-glaucoma agent, ophthalmic agent, anti-cholinergic, analgesic, anti-depressant, anti-psychotic, neurotoxin, hypnotic, tranquilizer, anti-convulsant, muscle relaxant, anti-Parkinson agent, anti-spasmodic, muscle contractant, channel blocker, miotic agent, anti-secretory agent, anti-thrombotic agent, anticoagulant, anti-cholinergic, β-adrenergic blocking agent, diuretic, cardiovascular active agent, vasoactive agent, vasodilating agent, anti-hypertensive agent, angiogenic agent, modulators of cell-extracellular matrix interactions (e.g. cell growth inhibitors and anti-adhesion molecules), or inhibitors/intercalators of DNA, RNA, protein-protein interactions, protein-receptor interactions. In certain embodiments, the inventive fusion proteins are administered in combination with an anti-cancer agent. In certain embodiments, the anti-cancer agent is cytarabine or daunorubicin.

In some embodiments, inventive pharmaceutical compositions may be administered in combination with any therapeutically active agent or procedure (e.g., surgery, radiation therapy) that is useful to treat, alleviate, ameliorate, relieve, delay onset of, inhibit progression of, reduce severity of, and/or reduce incidence of one or more symptoms or features of cancer.

### Methods of Use and Treatment

Provided herein is method of treating a disease or disorder, the method comprising administration of an inventive fusion protein to a subject in need thereof or a composition comprising the inventive fusion protein to a subject in need thereof. Also frovided herein are uses of a fusion protein as described herein for the manufacture of a medicament for use in treatment of a disease or disorder. Further provided herein is a fusion protein as described herein for use in treatment of a disease or disorder. Exemplary diseases, disorders, or conditions which may be treated by administration of an inventive fusion protein comprise proliferative, neurological, immunological, endocrinologic, cardiovascular, hematologic, and inflammatory diseases, disorders, or conditions, and conditions characterized by premature or unwanted cell death.

In certain embodiments, the proliferative disease includes, but is not limited to, cancer, hematopoietic neoplastic disorders, benign neoplasms (i.e., tumors), diabetic retinopathy, rheumatoid arthritis, macular degeneration, obesity, and atherosclerosis. In certain embodiments, the proliferative disease is cancer. In certain embodiments, the disease or disorder is associated with aberrant Hox activity. Aberrant Hox activity includes but are not limited to activities which are not normal such as mutations of Hox proteins or an aberrant Hox expression. In certain embodiments, the disease or disorder is cancer. In certain embodiments, the cancer is acute myeloid leukemia (AML). In certain embodiments, the cancer is breast cancer. In certain embodiments, the cancer is B cell-acute lymphoblastic leukemia (ALL). Other disease or disorder is associated with aberrant Hox activity are disorders of limb formation, such as hand-foot-genital syndrome, synpolydactyly (SPD), brachydactyly, hypodactyly; disorders of lung development such as bronchopulmonary sequestration and congenital cystic adenomatoid malformation; acquired disorders such as emphysema, primary pulmonary hypertension and lung carcinomas.

The fusion proteins are useful for the treatment of various cancers. Hox proteins have dual roles in cancer (see Shah & Sukumar (2010) Nat. Rev. Cancer. 10(5):361-71). Certain Hox proteins are overexpressed in certain tumors while others are underexpressed. Exemplary cancers associated with Hox proteins include but are not limited to oesophageal squamous cell carcinoma, lung carcinoma, neuroblastoma, ovarian carcinoma, cervical carcinoma, prostate carcinoma, and breast carcinoma. Thus, both fusion proteins with activator or repressor domains are useful as therapies for various cancers.

Exemplary cancers include, but are not limited to, carcinoma, sarcoma, or metastatic disorders, blood cancer, breast cancer, ovarian cancer including epithelial ovarian cancers, colon cancer, lung cancer, fibrosarcoma, myosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, gastric cancer, esophageal cancer, rectal cancer, pancreatic cancer, ovarian cancer, prostate cancer, uterine cancer, cancer of the head and neck, skin cancer, brain cancer, stomach cancer, squamous cell carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, and Kaposi's sarcoma.

In certain embodiments, the cancer is acute myeloid leukemia (AML). The fusion proteins are useful for treating AML because the are capable of repressing transcription at Hox-PBX DNA binding sites and countering the transcription activating properties of the Hoxa9-PBX-Meis1 complex. Thus, the fusion proteins are useful for causing cell differentiation in AML cells, thereby treating AML. The inventive fusion proteins are capable of modulating the transcription of a target gene such as SOX4, CD34, FLT3R, FOXP1, or DNAJC10. In certain embodiments, the fusion proteins are capable of repressing the transcription of one or more target genes. Repression of certain target genes by the fusion proteins causes cell differentiation and upregulates one or more differentiation-specific genes such as S100A8, myeloperoxidase, or neutrophil elastase. The fusion protein can be used to increase expression of myeloid differentiation markers such as Mac-1 or Gr-1.

In certain embodiments, the cancer is breast cancer. The inventive fusion proteins may be capable of upregulating levels of breast cancer genes such as BRACA1, thereby increasing disease latency and reducing breast cancer cell growth.

In certain embodiments, the cancer is B cell-acute lymphoblastic leukemia (ALL).

The E2A-PBX fusion protein results in B cell-acute lymphoblastic leukemia (ALL) in humans (Aspland, et al., The role of E2A-PBX1 in leukemogenesis. Oncogene (2001) 20(40):5708-5717). E2A-PBX recognizes TGATTGAT DNA sequence (PBX homodimer) and activates transcription. In certain embodiments, the fusion protein comprises a homodimeric HFP comprising PBX-PBX fusion with a transcription repressor domain, which should enable differentiation of B cell-ALL (using ER-E2A-PBX cells as a model).

In certain embodiments, the fusion proteins comprise a transcription activator domain. In certain embodiments, it may be desirable to prevent differentiation of certain cells such as hematopoietic stem cells (HSC). HSCs are rare stem cells that have the ability to differentiate into specialized blood cells, including lymphocytes, red blood cells, and platelets. While studies to successfully expand these cells have spanned over the last three decades, a routine method for ex vivo expansion of human HSCs is still not available. The inventive fusion proteins herein may enable the in vivo expansion of human HSCs.

In certain embodiments, the inventive fusion proteins is used as a monotherapy for AML. In certain embodiments, the inventive fusion proteins is used in combination with chemotherapy. In certain embodiments, the inventive fusion proteins is used in combination with standard or conventional AML treatment regimen (cytarabine, daunorubicin, idarubicin) given as a single agent. In certain embodiments, the fusion protein is used as single entity during the maintenance therapy period (post-induction therapy).

Over 206 homeodomain proteins are implicated in human diseases (see research.nhgri.nih.gov/homeodomain/?mode=like&view=disorders&sortby=ENTREZ_GEN E_SYMBOL). Transcription activator or repressor constructs utilizing truncated homeodomains may provide novel therapies for various diseases.

### General Methods

Libraries of homeodomain fusion protein (HFP) domains can be screened using yeast surface display, which can identify HFP domains exhibiting tight and specific binding to the desired DNA target site, such as the Hoxa9-Pbx1 DNA recognition site. Yeast surface display is further described in Boder and Wittrup, Yeast surface display for screening combinatorial polypeptide libraries, Nat Biotechnol 15, 553-7 (1997) and in U.S. Pat. No. 6,300,065. Generally, the yeast surface display method involves transforming a DNA library into cells (such as S. cerevisiae), in which the displayed proteins are fused to a yeast surface protein, Aga2p. Yeast cells are large enough to enable screening by fluorescence-activated cell sorting (FACS), which can evaluate >10⁷ cells per hour and is capable of sorting cells based on multiple fluorescent signals. This permits multiparameter sorting, allowing cells to be selected not based solely on their absolute binding (e.g., to a fluorescently labeled target protein) but based on ratios of different fluorophores.

Following the screening step, PCR-site selection can be used to confirm DNA-sequence selectivity of the HFP domains in vitro. The HFP domains can be isolated and purified from the yeast cell surface by enzymatic cleavage using, e.g., TEV cleavage. In vitro site selection PCR experiments can be performed using random DNA sequences. These steps allow HFP domains capabale of selective binding to the desired target site to be identified. In certain embodiments, one or more of the peptide domains of the fusion protein can be stapled or stitched, creating a cell-permeable fusion protein. Stapled or stitched peptides have been described in, for example, Walensky et al., Science (2004) 305:1466-1470; U.S. Pat. No. 8,592,377; U.S. Pat. No. 7,192,713; U.S. Patent Application Publication No. 2006/0008848; U.S. Patent Application Publication No. 2012/0270800; International Publication No. WO 2008/121767 and International Publication No. WO 2011/008260, each of which are incorporated herein by reference.

In vivo experiments using myeloid progenitors can be used to confirm that the HFP domains bind to the genomic DNA target site. Myeloid progenitors are treated with HFPs comprising a FLAG tag. Chromatin immunoprecipitation sequencing (ChIP-Seq) assays are then used to identify HFP domain candidates for cell differentiation studies.

Cellular assays such as a lysozyme-GFP myeloid progenitor cell assay can be used to test the ability of the fusion proteins to enable myeloid cell differentiation in vitro. Such cell lines can be useful for LFN-fusion proteins. Other cell lines useful for testing the fusion proteins include human AML lines, e.g., MOLM-14, THP-1, U937, HL60. Transcript levels of target genes can be measured by quantitative real time PCR.

In vivo testing of the fusion proteins can be performed using, e.g., murine models wherein MLL-AF9 transduced bone marrow cells are transplanted into mice. MLL-AF9 is a fusion oncoprotein in human AML and MLL-driven AMLs are critically dependent on Hoxa9 activity.

If adequate PCR-site selectivity for the Hoxa9-PBX DNA recognition sequence is not observed for HFPs, error-prone PCR can be used to introduce random mutations in focused HFP yeast libraries and directed evolution can be used to achieve desired selectivity for the DNA sequence. If sufficient nuclear localization is not achieved then a short nuclear localization sequence (PKKKRKV; SEQ ID NO: 19) will be included during the synthesis of HFPs.

Fusion proteins without amino acids for stapling or stitching can be prepared recombinantly using known methods. For example, recombinant protein expression of the fusion proteins or the LFN-fusion proteins can be performed using bacterial expression or other recombinant expression method such as in vitro translation, eukaryotic expression, insect culture. The histidine-tagged proteins can be purified by NTA-resin and tested in culture and in vivo if promising. The LFN-HFP fusions in vitro (cell culture) are tested in vitro using wild type-PA protein which can form pores in any cell type, to identify differentiation inducing activity. For in vivo studies, the mutant PA-scFv fusions to target AML cells specifically can be used to enable selective delivery of the LFN-HFP.

Fusion proteins comprising a stapled or stitched domain are prepared using standard peptide synthesis. Various methods known in the art can be used to fuse LFN to the fusion proteins comprising a stapled or stitched domain. For example, click chemistry ligation, native chemical ligation, and sortase-mediated ligation can be used fuse LFN to stapled fusion proteins and/or to fuse various domains of the the stapled fusion protein (e.g., attachment of stapled PBX domain to stapled Hox domain and/or stapled SID domain containing 3XNLS).

### Exemplary Fusion Proteins

Provided below are exemplary fustion proteins that have been prepared:

S1:

(SEQ ID NO: 28)

MATAVGMNIQLLLEAADYLERREREAEHGYASMLPYDPKKKRKVDPKKKR
KVDPKKKRKVGGDPERQVKIWFQNRRMKMKKINGDPVSQVSNWFGNKRIR
YKKNIG
S2:

(SEQ ID NO: 29)

MATAVGMNIQLLLEAADYLERREREAEHGYASMLPYDPKKKRKVDPKKKR
KVDPKKKRKVGGDPERQVKIWFQNRRMKMKKINGGDPVSQVSNWFGNKRI
RYKKNIG
S3:

(SEQ ID NO: 30)

MATAVGMNIQLLLEAADYLERREREAEHGYASMLPYDPKKKRKVDPKKKR
KVDPKKKRKVGGDPERQVKIWFQNRRMKMKKINGGGDPVSQVSNWFGNKR
IRYKKNIG
S4:

(SEQ ID NO: 31)

MATAVGMNIQLLLEAADYLERREREAEHGYASMLPYDPKKKRKVDPKKKR
KVDPKKKRKVGGDPERQVKIWFQNRRMKMKKINGGGGDPVSQVSNWFGNK
RIRYKKNIG
S2 mutant:

(SEQ ID NO: 32)

DPERQVKAWFAARRAKMKKINGGDPVSQVSEAWFGAKRIAYKKNIG
S3 mutant:

(SEQ ID NO: 33)

DPERQVKAWFAARRAKMKKINGGGDPVSQVSAWFGAKRIAYKKNIG

In certain embodiments, the fusion protein comprises the sequence of SEQ ID NO: 28. In certain embodiments, the fusion protein comprises the sequence of SEQ ID NO: 29. In certain embodiments, the fusion protein comprises the sequence of SEQ ID NO: 30. In certain embodiments, the fusion protein comprises the sequence of SEQ ID NO: 31. In certain embodiments, the fusion protein comprises the sequence of SEQ ID NO: 32. In certain embodiments, the fusion protein comprises the sequence of SEQ ID NO: 33. Any of the foregoing precent homology and percent identity embodiments described herein are applicable to SEQ ID NO: 28 to 33.

Provided below in Table I are domain schematics of the exemplary fusion proteins to illustrate certain embodiments of the invention. Fusion proteins S3 and 1-10 below contain a C-terminal 3XFLAG (not shown), with a GG linker between the Flag tag and the HFP domain, Fusion protein 10 is a N-terminal 3XFLAG separated by a GG linker from the HFP domain. Each instance of “N” indicates a nuclear localization sequence (DPKKKRKV, SEQ ID NO: 18). “DPA” is the DPA alpha-helix nucleating motif sequence. “Hoxa9-G3-PBX” is the homeodomain fusion protein (HFP) domain comprising the HoxA9 and PBX truncated homeodomains with a GGG linker fusing the two homeodomains and with a DP alpha helix nucleating motif used at the N-terminal side of each Hoxa9 and PBX helices: DPERQVKIWFQNRRMKMKKINGGGDPVSQVSNWFGNKRIRYKKNIG (SEQ ID NO: 34)

TABLE 1

| | SEQ ID | | |
|---|---|---|---|
| Ref. no. | NO: | Domain arrangement | Activity |
| S3 | 11 | 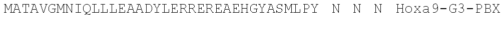 | ✓ |
| 1 | 11 | 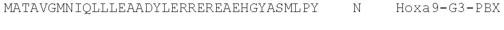 | ✓ |
| 2 | 11 | 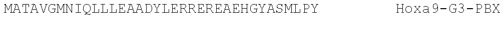 | x |
| 3 | 12 | 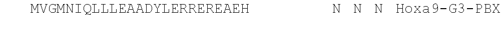 | ✓ |
| 4 | 13 | 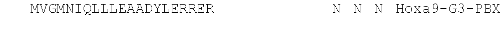 | ✓ |
| 5 | 14 | 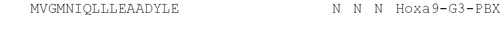 | x |
| 6 | 15 | 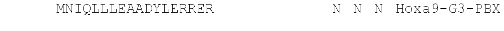 | x |
| 7 | 16 | 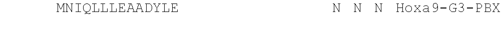 | x |
| 8 | 17 | 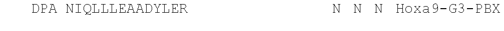 | x |
| 9 | 17 | 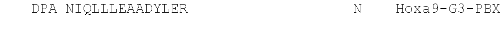 | x |
| 10 | 11 | 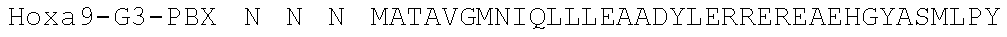 | ✓ |

The amino acid sequences for fusion proteins numbers S3 and 1-10 are provided below. For fusion protein number 10, the SID sequence does not contain the first methionine since the SID sequence does not need the methionine for activity. Since Met is the start codon, it was often placed preceding the start of the HFP domain. In addition, Ala can be incorporated following the Met so that proximity of Met does not interfere with the ucleating activity of DP. Also note that the 3XFLAG tag (not shown) is not an active component of the fusion proteins and is an experimental tool used to enable checking genomic DNA binding sites and assess sequence specificity (for Hox/PBX DNA targets) by ChiP-seq or Chip-PCR to be conducted. The final active versions of the fusion proteins can be with or without the FLAG tag. Other tags in addition to FLAG and 3XFLAG that may be used to determine DNA-target specificity in whole cells are HA-tag or myc-tag.

S3 = Full SID-3XNLS-HFP

(SEQ ID NO: 35)

MATAVGMNIQLLLEAADYLERREREAEHGYASMLPYDPKKKRKVDPKKKR
KVDPKKKRKVGGDPERQVKIWFQNRRMKMKKINGGGDPVSQVSNWFGNKR
IRYKKNIG.
1 = Full SID-1XNLS-HFP:

(SEQ ID NO: 36)

MATAVGMNIQLLLEAADYLERREREAEHGYASMLPYDPKKKRKVGGDPER
QVKIWFQNRRMKMKKINGGGDPVSQVSNWFGNKRIRYKKNIG.
2 = Full SID-no NLS-HFP:

(SEQ ID NO: 37)

MATAVGMNIQLLLEAADYLERREREAEHGYASMLPYGGDPERQVKIWFQN
RRMKMKKINGGGDPVSQVSNWFGNKRIRYKKNIG.
3 = SID-3XNLS-HFP:

(SEQ ID NO: 38)

MVGMNIQLLLEAADYLERREREAEHGGDPKKKRKVDPKKKRKVDPKKKRK
VGGDPERQVKIWFQNRRMKMKKINGGGDPVSQVSNWFGNKRIRYKKNIG.
4 = SID-3XNLS-HFP:

(SEQ ID NO: 39)

MVGMNIQLLLEAADYLERRERGSDPKKKRKVDPKKKRKVDPKKKRKVGGD
PERQVKIWFQNRRMKMKKINGGGDPVSQVSNWFGNKRIRYKKNIG.
5 = SID-3XNLS-HFP:

(SEQ ID NO: 40)

MVGMNIQLLLEAADYLEGGDPKKKRKVDPKKKRKVDPKKKRKVGGDPERQ
VKIWFQNRRMKMKKINGGGDPVSQVSNWFGNKRIRYKKNIG.
6 = SID₈₋₂₄-3XNLS-HFP:

(SEQ ID NO: 41)

MNIQLLLEAADYLERRERGGDPKKKRKVDPKKKRKVDPKKKRKVGGDPER
QVKIWFQNRRMKMKKINGGGDPVSQVSNWFGNKRIRYKKNIG.
7 = SID₈₋₂₀-3XNLS-HFP:

(SEQ ID NO: 42)

MNIQLLLEAADYLEGGDPKKKRKVDPKKKRKVDPKKKRKVGGDPERQVKI
WFQNRRMKMKKINGGGDPVSQVSNWFGNKRIRYKKNIG.
8 = DPA-SID₈₋₂₁-3XNLS-HFP:

(SEQ ID NO: 43)

MADPANIQLLLEAADYLERGGDPKKKRKVDPKKKRKVDPKKKRKVGGDPE
RQVKIWFQNRRMKMKKINGGGDPVSQVSNWFGNKRIRYKKNIG.
9 = DPA-SID₈₋₂₁-1XNLS-HFP:

(SEQ ID NO: 44)

MADPANIQLLLEAADYLERGGDPKKKRKVGGDPERQVKIWFQNRRMKMKK
INGGGDPVSQVSNWFGNKRIRYKKNIG.
10 = HFP-3XNLS-Full SID:

(SEQ ID NO: 45)

DPERQVKIWFQNRRMKMKKINGGGDPVSQVSNWFGNKRIRYKKNIGGGDP
KKKRKVDPKKKRKVDPKKKRKVATAVGMNIQLLLEAADYLERREREAEHG
YASMLPY.
S3 mutant:

(SEQ ID NO: 46)

MATAVGMNIQLLLEAADYLERREREAEHGYASMLPYDPKKKRKVDPKKKR
KVDPKKKRKVGGDPERQVKAWFAARRAKMKKINGGGDPVSQVSAWFGAKR
IAYKKNIG.

Provided below in Table 2 are domain schematics for an LFN sequence fused to the fusion proteins (LFN-fusion proteins). The fusion proteins will be tested for activity. LFN refers to the N-terminal portion of anthrax toxin lethal factor (LF_{N}) of SEQ ID NO: 25. The LFN-fusion proteins in Table 2 contain a SGGGGS (SEQ ID NO: 57) linker between the LFN domain and the rest of the fusion protein. in Table 2, the domains 3xNLS-LFN has a LFN sequence with an NLS sequence embedded into the LFN sequence. We are yet to test the functionality and cellular localization of these newly-created LFN-REP proteins.

TABLE 2

| Ref. | |
|---|---|
| No. | Domain arrangement |
| 12 | 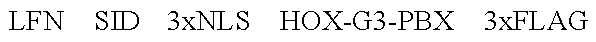 |
| 13 | 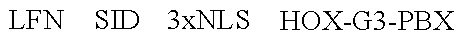 |
| 14 | 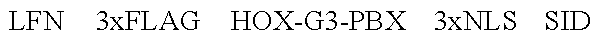 |
| 15 | 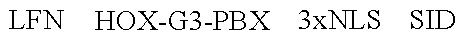 |
| 16 | 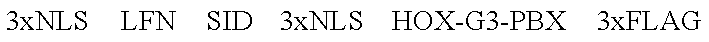 |
| 17 | 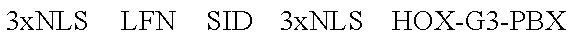 |
| 18 | 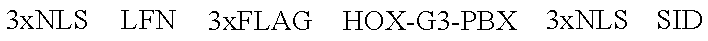 |
| 19 | 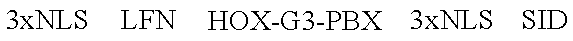 |

12 = LFN-SID-3XNLS-HFP-3XFLAG

(SEQ ID NO: 47)

MGSSHHHHHHSSGLVPRGSHMAGGHGDVGMHVKEKEKNKDENKRKDEERN
KTQEEHLKEIMKHIVKIEVKGEEAVKKEAAEKLLEKVPSDVLEMYKAIGG
KIYIVDGDITKHISLEALSEDKKKIKDIYGKDALLHEHYVYAKEGYEPVL
VIQSSEDYVENTEKALNVYYEIGKILSRDILSKINQPYQKFLDVLNTIKN
ASDSDGQDLLFTNQLKEHPTDFSVEFLEQNSNEVQEVFAKAFAYYIEPQH
RDVLQLYAPEAFNYMDKFNEQEINLSLEELKDQRSGRELESGGGGSMATA
VGMNIQLLLEAADYLERREREAEHGYASMLPYDPKKKRKVDPKKKRKVDP
KKKRKVGGDPERQVKIWFQNRRMKMKKINGGGDPVSQVSNWFGNKRIRYK
KNIGGGDYKDHDGDYKDHDIDYKDDDDK.
13 = LFN-SID-3XNLS-HFP

(SEQ ID NO: 48)

MGSSHHHHHHSSGLVPRGSHMAGGHGDVGMHVKEKEKNKDENKRKDEERN
KTQEEHLKEIMKHIVKIEVKGEEAVKKEAAEKLLEKVPSDVLEMYKAIGG
KIYIVDGDITKHISLEALSEDKKKIKDIYGKDALLHEHYVYAKEGYEPVL
VIQSSEDYVENTEKALNVYYEIGKILSRDILSKINQPYQKFLDVLNTIKN
ASDSDGQDLLFTNQLKEHPTDFSVEFLEQNSNEVQEVFAKAFAYYIEPQH
RDVLQLYAPEAFNYMDKFNEQEINLSLEELKDQRSGRELESGGGGSMATA
VGMNIQLLLEAADYLERREREAEHGYASMLPYDPKKKRKVDPKKKRKVDP
KKKRKVGGDPERQVKIWFQNRRMKMKKINGGGDPVSQVSNWFGNKRIRYK
KNIG.
14 = LFN-3XFLAG-HFP-3XNLS-SID

(SEQ ID NO: 49)

MGSSHHHHHHSSGLVPRGSHMAGGHGDVGMHVKEKEKNKDENKRKDEERN
KTQEEHLKEIMKHIVKIEVKGEEAVKKEAAEKLLEKVPSDVLEMYKAIGG
KIYIVDGDITKHISLEALSEDKKKIKDIYGKDALLHEHYVYAKEGYEPVL
VIQSSEDYVENTEKALNVYYEIGKILSRDILSKINQPYQKFLDVLNTIKN
ASDSDGQDLLFTNQLKEHPTDFSVEFLEQNSNEVQEVFAKAFAYYIEPQH
RDVLQLYAPEAFNYMDKFNEQEINLSLEELKDQRSGRELESGGGGSMDYK
DHDGDYKDHDIDYKDDDDKGGDPERQYKIWRINRRMKNAKKINGGGDPVS
QVSNWFGNKRIRYKKNIGGGDPKKKRKVDPKKKRKVDPKKKRKVATAVGM
NIQLLLEAADYLERREREAEHGYASMLPY.
15 = LFN-HFP-3XNLS-SID

(SEQ ID NO: 50)

MGSSHHHHHHSSGLVPRGSHMAGGHGDVGMHVKEKEKNKDENKRKDEERN
KTQEEHLKEIMKHIVKIEVKGEEAVKKEAAEKLLEKVPSDVLEMYKAIGG
KIYIVDGDITKHISLEALSEDKKKIKDIYGKDALLHEHYVYAKEGYEPVL
VIQSSEDYVENTEKALNVYYEIGKILSRDILSKINQPYQKFLDVLNTIKN
ASDSDGQDLLFTNQLKEHPTDFSVEFLEQNSNEVQEVFAKAFAYYIEPQH
RDVLQLYAPEAFNYMDKFNEQEINLSLEELKDQRSGRELESGGGGSMADP
ERQVKIWFQNRRMKMKKINGGGDPVSQVSNWFGNKRIRYKKNIGGGDPKK
KRKVDPKKKRKVDPKKKRKVATAVGMNIQLLLEAADYLERREREAEHGYA
SMLPY.
16 = 3XNLS/LFN-SID-3XNLS-HFP-3XFLAG

(SEQ ID NO: 51)

MGSSHHHHHHSSGLVPRGSDPKKKRKVDPKKKRKVDPKKKRKVGGHMAGG
HGDVGMHVKEKEKNKDENKRKDEERNKTQEEHLKEIMKHIVKIEVKGEEA
VKKEAAEKLLEKVPSDVLEMYKAIGGKIYIVDGDITKHISLEALSEDKKK
IKDIYGKDALLHEHYVYAKEGYEPVLVIQSSEDYVENTEKALNVYYEIGK
ILSRDILSKINQPYQKFLDVLNTIKNASDSDGQDLLFTNQLKEHPTDFSV
EFLEQNSNEVQEVFAKAFAYYIEPQHRDVLQLYAPEAFNYMDKFNEQEIN
LSLEELKDQR [symbol] SGGGGSMATAVGMNIQLLLEAADYLERREREAEHGYAS
MLPYDPKKKRKVDPKKKRKVDPKKKRKVGGDPERQVKIWFQNRRMKMKKI
NGGGDPVSQVSNWFGNKRIRYKKNIGGGDYKDHDGDYKDHIDYKDDDD
IK.
17 = 3xNLS/LFP-SID-3XNLS-HFP

(SEQ ID NO: 52)

MGSSHHHHHHSSGLVPRGSDPKKKRKVDPKKKRKVDPKKKRKVGGHMAGG
HGDVGMHVKEKEKNKDENKRKDEERNKTQEEHLKEIMKHIVKIEVKGEEA
VKKEAAEKLLEKVPSDVLEMYKAIGGKIYIVDGDITKHISLEALSEDKKK
IKDIYGKDALLHEHYVYAKEGYEPVLVIQSSEDYVENTEKALNVYYEIGK
ILSRDILSKINQPYQKFLDVLNTIKNASDSDGQDLLFTNQLKEHPTDFSV
EFLEQNSNEVQEVFAKAFAYYIEPQHRDVLQLYAPEAFNYMDKFNEQEIN
LSLEELKDQRSGRELESGGGGSMATAVGMNIQLLLEAADYLERREREAEH
GYASMLPYDPKKKRKVDPKKKRKVDPKKKRKVGGDPERQVKIWFQNRRMK
MKKINGGGDPVSQVSNWFGNKRIRYKKNIG.
18 = 3XNLS/LFN-3XFLAG-HFP-3XNLS-SID

(SEQ ID NO: 53)

MGSSHHHHHHSSGLVPRGSDPKKKRKVDPKKKRKVDPKKKRKVGGHMAGG
HGDVGMHVKEKEKNKDENKRKDEERNKTQEEHLKEIMKHIVKIEVKGEEA
VKKEAAEKLLEKVPSDVLEMYKAIGGKIYIVDGDITKHISLEALSEDKKK
IKDIYGKDALLHEHYVYAKEGYEPVLVIQSSEDYVENTEKALNVYYEIGK
ILSRDILSKINQPYQKFLDVLNTIKNASDSDGQDLLFTNQLKEHPTDFSV
EFLEQNSNEVQEVFAKAFAYYIEPQHRDVLQLYAPEAFNYMDKFNEQEIN
LSLEELKDQRSGRELESGGGGSMDYKDHDGDYKDHDIDYKDDDDKGGDPE
RQVKIWFQNRRMKMKKINGGGDPVSQVSNWFGNKRIRYKKNIGGGDPKKK
RKVDPKKKRKVDPKKKRKVATAVGMNIQLLLEAADYLERREREAEHGYAS
MLPY.
19 = 3XNLS/LFN-HFP-3XNLS-SID

(SEQ ID NO: 54)

MGSSHHHHHHSSGLVPRGSDPKKKRKVDPKKKRKVDPKKKRKVGGHMAGG
HGDVGMHVKEKEKNKDENKRKDEERNKTQEEHLKEIMKHIVKIEVKGEEA
VKKEAAEKLLEKVPSDVLEMYKAIGGKIYIVDGDITKHISLEALSEDKKK
IKDIYGKDALLHEHYVYAKEGYEPVLVIQSSEDYVENTEKALNVYYEIGK
ILSRDILSKINQPYQKFLDVLNTIKNASDSDGQDLLFTNQLKEHPTDFSV
EFLEQNSNEVQEVFAKAFAYYIEPQHRDVLQLYAPEAFNYMDKFNEQEIN
LSLEELKDQR [symbol] SGGGGSMADPERQVKIWFQNRRMKMKKINGGGDP
VSQVSNWFGNKRIRYKKNIGGGDPKKKRKVDPKKKRKVDPKKKRKVATAV
GMNIQLLLEAADYLERREREAEHGYASMLPY.

Provided below in Table 3 are exemplary LFN-fusion proteins that can be constructed using full-length homeodomains for HoxA9 and PBX. The NLS domain can be varied to have one NLS to three NLS. The SID sequence can be replaced with KRAB sequence or variant thereof. The linker SGGGGSGGGGS (SEQ ID NO: 58) can be replaced with other linkers of varying lengths and/or composition.

TABLE 3

| | SEQ | |
|---|---|---|
| Ref | ID | |
| No. | NO. | Domain arrangement |
| 20 | 58 | 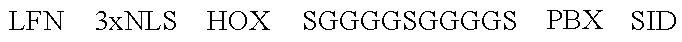 |
| 21 | 58 | 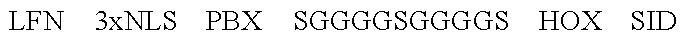 |

In certain embodiments, the fusion protein comprises the sequence of SEQ ID NO: 47. In certain embodiments, the fusion protein comprises the sequence of SEQ ID NO: 48. In certain embodiments, the fusion protein comprises the sequence of SEQ ID NO: 49. In certain embodiments, the fusion protein comprises the sequence of SEQ ID NO: 50. In certain embodiments, the fusion protein comprises the sequence of SEQ ID NO: 51. In certain embodiments, the fusion protein comprises the sequence of SEQ ID NO: 52. In certain embodiments, the fusion protein comprises the sequence of SEQ ID NO: 53. In certain embodiments, the fusion protein comprises the sequence of SEQ ID NO: 54. Any of the foregoing precent homology and percent identity embodiments described herein are applicable to SEQ ID NO: 47 to 54.

Cell-penetrating peptides can be used to make the fusion proteins cell-permeable. In certain embodiments, the fusion proteins can be attached to a cell-penetrating peptide. In certain embodiments, the cell-penetrating peptide is TAT. The sequence for TAT is YGRKKRPQRRR (SEQ ID NO: 59). In certain embodiments, the cell-penetrating peptide is selective for the specific target cell (e.g., target specific tumor cell types) for delivering the therapeutic fusion peptide. In certain embodiments, the cell-penetrating peptide is selective for AML cells. In certain embodiments, the cell-penetrating peptide is CPP44. The sequence for CPP44 is KRPTMRFRYTWNPMK (SEQ ID NO: 60). Tumour lineage-homing cell-penetrating peptides are described, e.g., in PCT Application WO 2011/126010, incorporated herein by reference in its entirety.

It is understood that the foregoing detailed description and the following examples are illustrative only and are not to be taken as limitations upon the scope of the invention. Various changes and modifications to the disclosed embodiments, which will be apparent to those skilled in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### EXAMPLES

In order that the invention described herein may be more fully understood, the following examples are set forth. It should be understood that these examples are for illustrative purposes only and are not to be construed as limiting this invention in any manner.

### Example 1

### Develop Truncated Homeodomain Fusion Proteins (HFPs) that Bind to a Target DNA Sequence

Fusion protein constructs were initially screened using a yeast display library. The yeast display library was prepared through homologous recombination in yeast to introduce diversity in the linker and create libraries with linker lengths varying between 1-4 residues between the Hox and PBX helices. We attempted to screen the library using double stranded DNA that is labelled with a fluorophore and contains the Hox/PBX DNA recognition site (5′ to 3′ TGATTTAC or TGATTTAT). The screen may be done in the presence of excess non-specific DNA that is not fluorescently labelled so as to identify clones specific for the target sequence.

Analysis of the crystal structure of HoxA9-Pbx1-DNA complex revealed the C-terminus of the HoxA9 DNA binding helix is in close proximity to the N-terminus of the Pbx1 DNA binding helix with both helices interacting with the major groove (FIG. 1A).(8) We designed fusion proteins comprising homeodomain fusion proteins of HoxA9 and PBX DNA recognition helices having the same or similar DNA sequence specificity as the full-length Hoxa9-PBX heterodimer complex. In order to develop fusion proteins capable of sequence selective DNA recognition of the Hoxa9-Pbx1 DNA consensus site, we constructed a yeast surface display library of fused truncated homeodomains of Hoxa9 and Pbx1 (>10⁷ transformants).(9) To facilitate a-helicity of the truncated (20 amino acids long) homeodomains, we placed a strong α-helix nucleating aspartic acid-proline (DP) motif at the N-terminus of Hoxa9 and Pbx1 truncated sequences.(10) We varied the linker length (X=1 to 4) between the two helices and included randomization (FIG. 1A) at positions (as suggested by in silico modeling) within the helix expected to play a role in stabilizing the desired helix-turn-helix conformation.

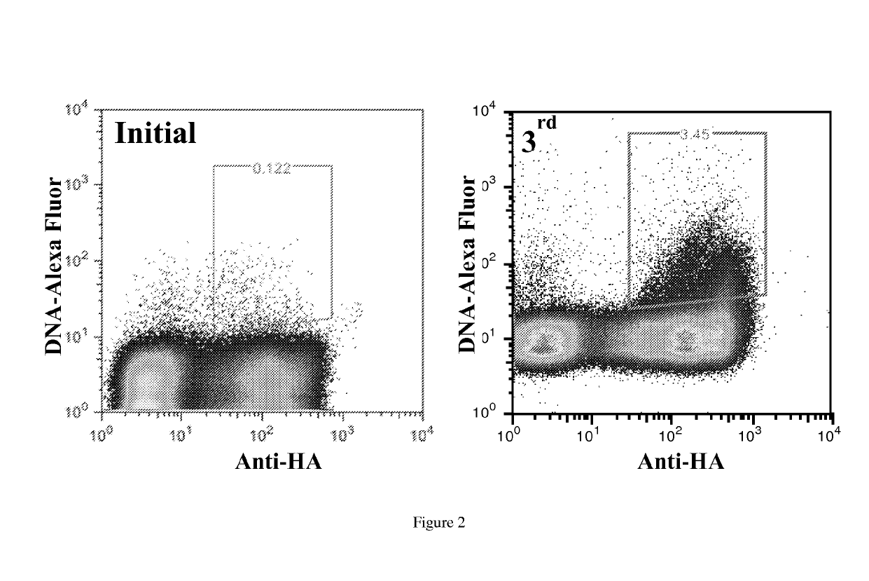

The fusion proteins were screened using yeast surface display library by fluorescence activated cell sorting (FIG. 2) for selective binding to fluorescently-labeled Hoxa9-Pbx1 DNA recognition sequence (TGATTTAC) in the presence of 10-fold excess randomized DNA (TAGTCATT). Hemagglutinin (HA) tag detection at the N-terminus was used to normalize for protein expression and reduce false positives. As shown in FIG. 2, enrichment for binders to the desired DNA sequence was observed after the third round of sorting.

To repress transcription at the desired desired genomic loci through histone deacetylation, concise transcription repressor domains were developed that can be fused to homeodomain fusion protein domains. Since the fusion proteins are short and consist of 3 distinct α-helices, peptide stapling is one approach to create a cell-permeable DNA-targeting therapy. The repressor domains are based on Sin3-interacting domain (SID) of Mad1 protein. SID is a 25 amino acid α-helix containing motif that recruits Sin3, HDAC1 and HDAC2 and inhibits transcription in whole cells by histone modification.(13, 23) Transcription silencing in whole cells has been observed upon fusion of the 25 amino acid SID is fused to ectopically-expressed DNA-targeting proteins.(24) Previously, using peptide stapling technology to stabilize the α-helix in SID, SID peptide was truncated to 17 amino acids. This truncated stapled peptide version displayed increased affinity for Sin3 (Kd=10 nM) versus the wild-type SID (Kd=70 nM), and exhibited cell and nuclear permeability in live cells. Repressor domains such as SID, including the stapled version, can be fused to homeodomain fusion protein domains.

Nucleic acid constructs encoding the fusion proteins were designed and prepared through gene synthesis (IDT DNA) and cloned into retroviral vectors or bacterial expression vectors. All the AML cell data is from retroviral expression of the constructs in a murine stem cell virus (MSCV) vector. AML model cells, dependent on Hoxa9 and Meis1, were created using murine progenitor cells transduced with Hoxa9 and Meis1. Using a murine stem cell virus (MSCV) retroviral vector containing an IRES GFP, fusion proteins such as the one shown in FIG. 2, were introduced into and ectopically expressed in the aggressive Hoxa9-Meis1 immortalized AML model cells. At about 3 days post-transduction, Hoxa9-Meis1 cells were sorted for cells which were GFP positive for the fusion proteins, expanded and 200,000 cells transplanted 10 days after transduction into sub-lethally irradiated mice (4.5 Gy), 5 mice per group. From data taken from percent GFP positive cell measurements and GFP ratios in cell, it was observed that there was a decline in repressor cells (repressor construct had growth disadvantage). Stable cells with low expression levels were selected for by Day 10. The cells were cultured for an additional 30 days after which the cell differentiation status was determined by flow cytometry after staining for myeloid differentiation markers such as Mac-1 (CD11b), Gr-1 and FLT3R expression. Genes were also assessed using QPCR on various days such as day 10, 20, or 30. Wright-Giemsa morphology staining was performed of the cells. On day 10, cells containing the fusion proteins were transplanted into mice. The latency of disease was then assessed. The spleen and bone marrow cells were frozen and analyzed for GFP, CD45.1, or CD33. Cell surface markers, such as c-Kit, Flt3, Mac-1, or Gr-1, were also analyzed.

Biological activity testing was conducted using fusion proteins S1 to S4 with various glycine linkers ranging from 1-4 glycines in length. S1 to S4 have the general domain arrangement of SID-3XNLS-GG linker-HoxA9-(G)₁₋₄-PBX wherein the glycine linker between the HoxA9 and PBX domain are varied from 1 to 4 glycines. A 3XFLAG tag was also included at the C-terminus but is not shown below in the sequences of S1 to S4.

Si:

(SEQ ID NO: 28)

MATAVGMNIQLLLEAADYLERREREAEHGYASMLPYDPKKKRKVDPKKKR
KVDPKKKRKVGGDPERQVKIWFQNRRMKMKKINGDPVSQVSNWFGNKRIR
YKKNIG
S2:

(SEQ ID NO: 29)

MATAVGMNIQLLLEAADYLERREREAEHGYASMLPYDPKKKRKVDPKKKR
KVDPKKKRKVGGDPERQVKIWFQNRRMKMKKINGGDPVSQVSNWFGNKRI
RYKKNIG
S3:

(SEQ ID NO: 30)

MATAVGMNIQLLLEAADYLERREREAEHGYASMLPYDPKKKRKVDPKKKR
KVDPKKKRKVGGDPERQVKIWFQNRRMKMKKINGGGDPVSQVSNWFGNKR
IRYKKNIG
S4:

(SEQ ID NO: 31)

MATAVGMNIQLLLEAADYLERREREAEHGYASMLPYDPKKKRKVDPKKKR
KVDPKKKRKVGGDPERQVKIWFQNRRMKMKKINGGGGDPVSQVSNWFGNK
RIRYKKNIG

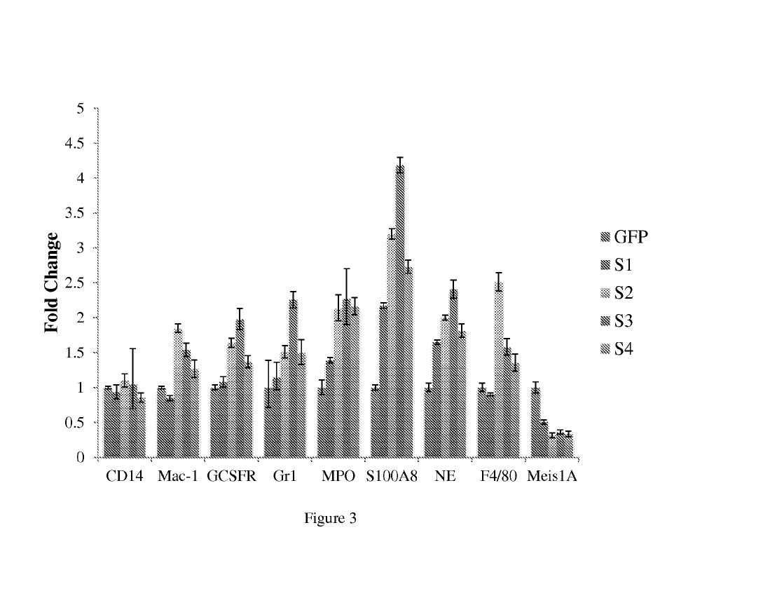

FIG. 3 shows that the S3 construct typically results in higher mRNA levels of differentiation-specific markers

A cell survival study was also conducted using fusion proteins S1-S4. Table 4 below shows that Hoxa9-Meis1 cells that express S3 demonstrated longer latency in vivo that was statistically significant.

TABLE 4

| | Median Survival | Log Rank | |
|---|---|---|---|
| Group | days | p value | Signifcant? |
| GFP | 62 | — | — |
| S1 | 62 | 0.656 | No |
| S2 | 67 | 0.261 | No |
| S3 | 94 | 0.002 | Yes |
| S4 | 69 | 0.271 | No |

Mutants of S2 and S3 were also constructed. The mutants have an HFP domain comprising the following sequence with DNA base contact residues mutated to alanine (underlined):

S2 mutant:

(SEQ ID NO: 32)

DPERQVKAWFAARRAKMKKINGGDPVSQVSAWFGAKRIAYKKNIG
S3 mutant:

(SEQ ID NO: 33)

DPERQVKAWFAARRAKMKKINGGGDPVSQVSAWFGAKRIAYKKNIG

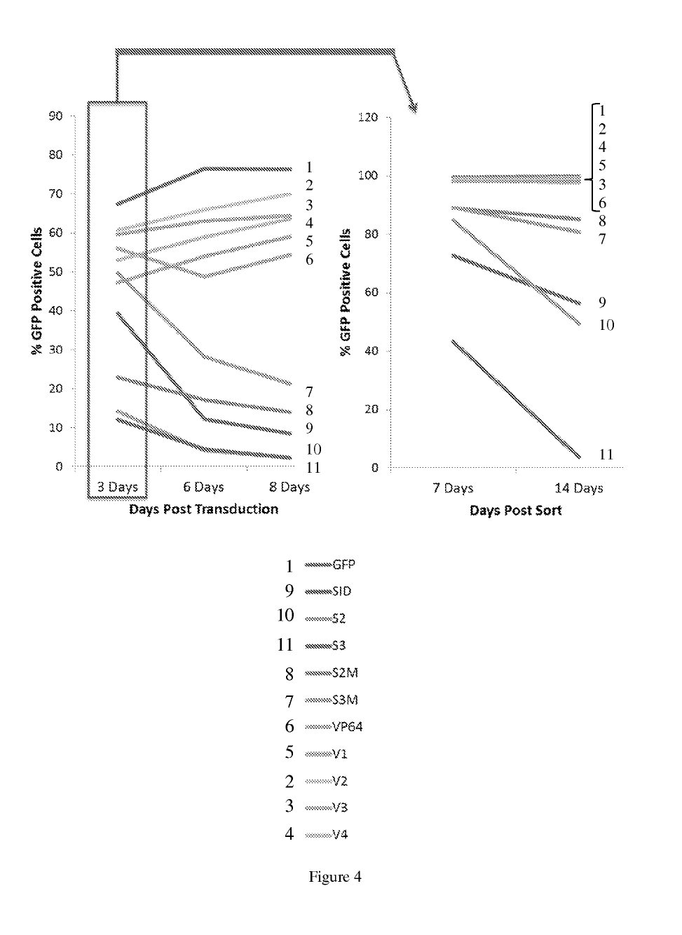

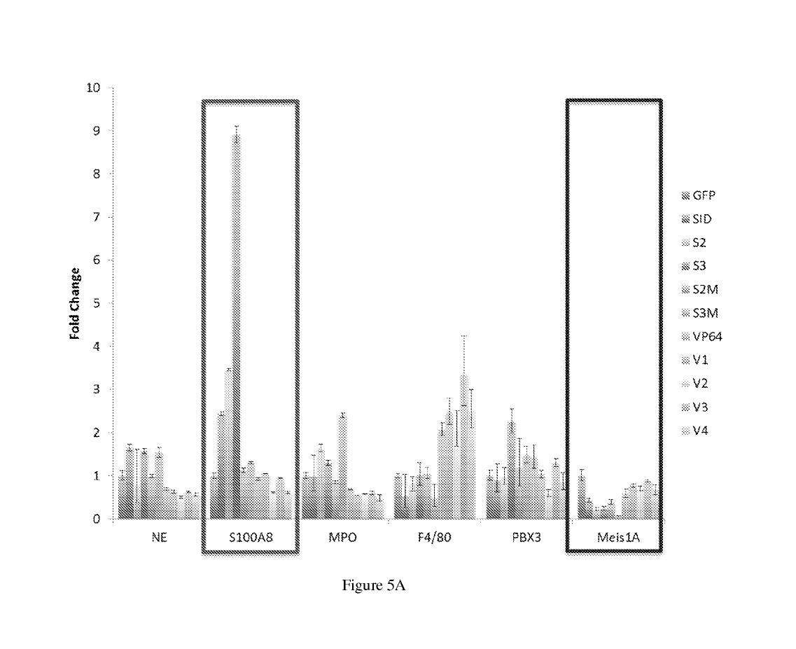

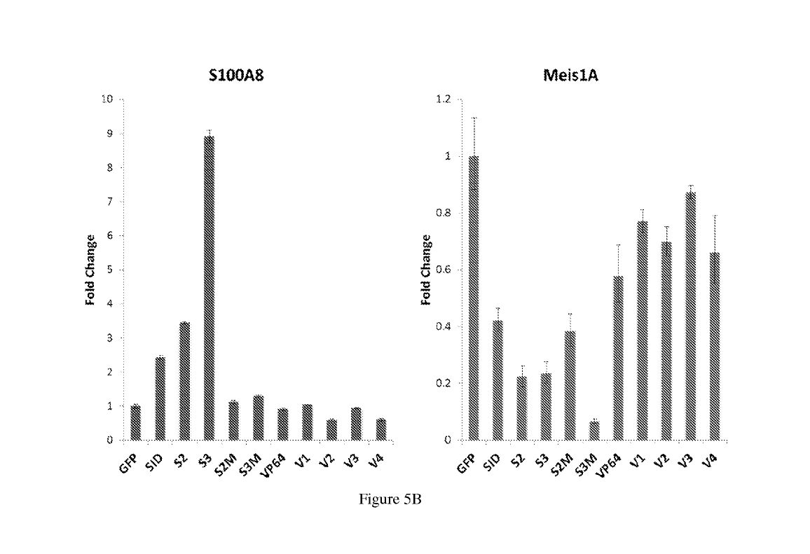

A construct comprising only the SID-3XNLS-3XFLAG was constructed (labeled as “SID” in FIGS. 4 and 5A-B).

Four additional constructs (V1-V4) containing the same HFP domain as those in S1-S4 but comprising a transcription activator domain were also prepared. The activator domain was prepared from 4x VP16. V1 has an HFP with a G linker; V2 has an HFP with a GG linker; V3 has an HFP with a GGG linker; V4 has an HFP with a GGGG (SEQ ID NO: 55) linker. A construct comprising only the 3XFLAG-3XNLS-VP64 was constructed (labeled “VP64” in FIGS. 4 and 5). The growth phenotype of the SID control, S2, S3, S2 mutant, S3 mutant, VP64 control, V1 to V4 are shown in FIGS. 4. S2 and S3 show more growth deficit than the corresponding S2 and S3 mutants.

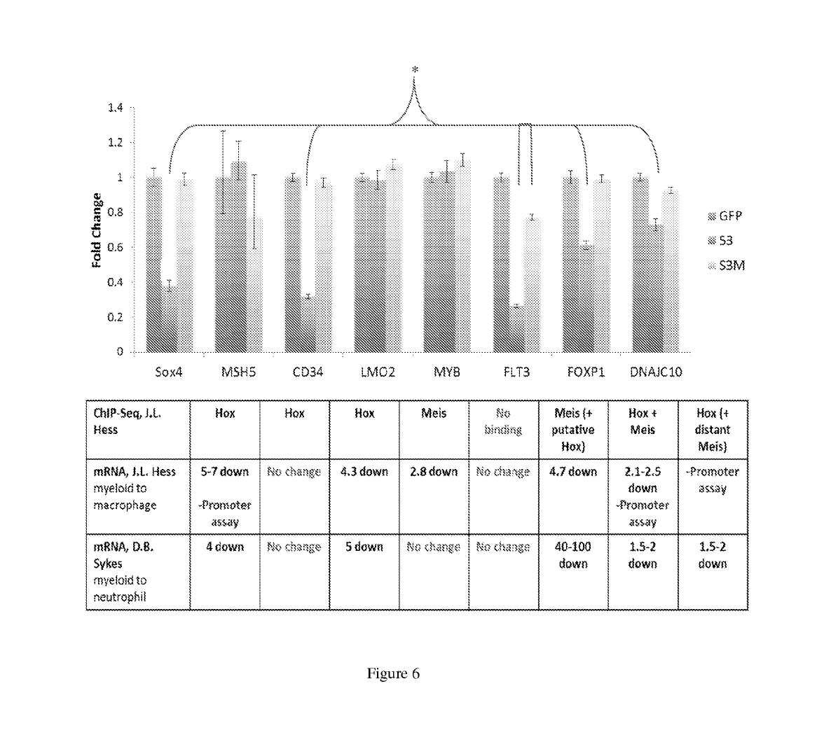

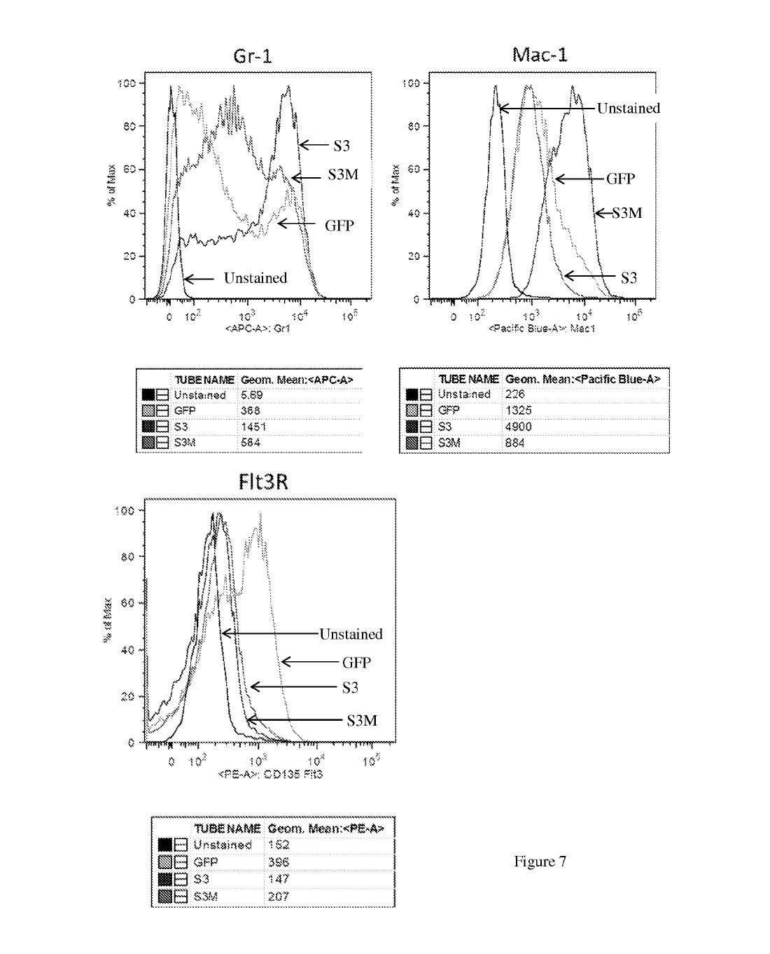

FIG. 5A is a QPCR of Mutants and Wild-type Constructs on day 17 in Hoxa9-Meis1 cells. FIG. 5B is an expanded view of the QPCR data for the S100A8 and Meis1A markers. FIG. 6 shows the Q-PCR of Direct Hoxa9 or Meis1 Targets in Hoxa9/Meis1 Cells, 17 days after transduction. S3 suppresses multiple targets of Hoxa9 but the S3 mutant (S3M) does not. FIG. 7 shows the cell surface markers on day 30. Granulocyte Differentiation Markers Gr-1 and Mac-1 Expression increased whereas Flt3 Receptor Decreased.

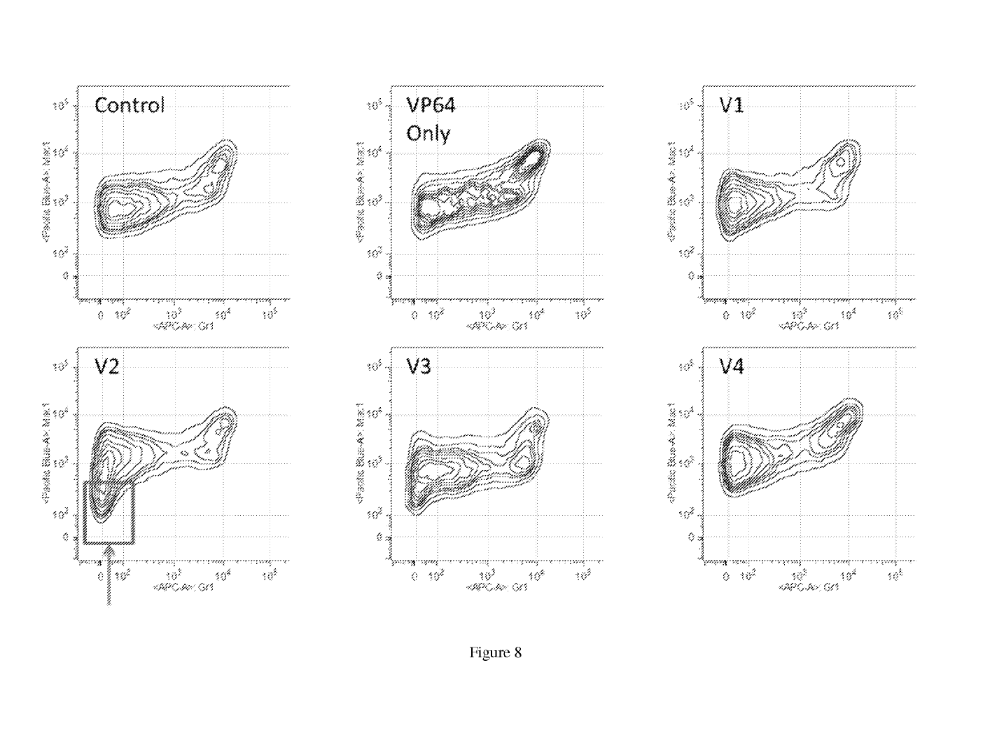

FIG. 8 shows activator constructs Meis1-Hoxa9 cells at 30 Days. Cells shift to a much more primitive state as they express less Gr-1 and Mac-1 (differentiation markers) than control. Such constructs may be useful for transient expansion of hematopoietic stem cells in vitro prior to transplantation or for restoring Hox activity in cancers where low Hoxa9 activity contributes to cancer e.g. breast cancer

In summary, constructs with GG or GGG linkers generally appear to be relatively more potent constructs; Meis1 transcripts are down regulated (possibly off-target effect); S100A8, MPO, NE, Mac-1, Gr-1 transcripts are elevated; Gr-1 protein expression is increased; Flt3 and c-Kit receptor protein expression are down-regulated; S2M and S3M (mutants) display less growth inhibition than S2 and S3; activator constructs have no growth defects.

Linker length and fusion protein composition optmization can be later performed using either yeast or phage display. Another screening approach that may be employed is a reporter assay in yeast or mamalian cells that are modified to express a fluorescent protein/luciferase under a custom promoter containing the TGATTTA(C/T) hox/pBX motif.

### Example 2

### Lysozyme-GFP Cell Based Assay to Monitor Cell Differentiation

A Lysozyme-GFP cell based assay is useful for investigating the ability of the fusion proteins to induce AML cell differentiation in culture and in vivo. For example, the LFN-fusion proteins can be tested using this type of assay. To develop a cell-based system to accurately model Hoxa9-mediated differentiation arrest in AML, a conditional version of Hoxa9 fused to the hormone binding domain of the estrogen receptor was introduced into bone marrow cells derived from a transgenic mouse in which green fluorescent protein (GFP) was expressed downstream of the endogenous lysozyme promoter. When these cells are cultured in the presence of β-estradiol and stem cell factor (SCF), the cells are arrested in myeloid differentiation and have the ability to proliferate indefinitely. As lysozyme is a secondary granule protein that is only expressed in differentiated myeloid cells, the undifferentiated cells are GFP negative when Hoxa9 was expressed, whereas inactivation of Hoxa9 (by removal of β-estradiol) resulted in 100% of cells becoming brightly GFP positive in just 4 days. Removal of beta-estradiol induced differentiation of the cells to mature neutrophils as shown by Wright-Giemsa morphology staining and expression of Gr-1 and Mac-1 cell surface markers.

### Example 3

### Methods to Prepare and Characterize Stapled Fusion Proteins

For fusion proteins small enough (about 40-45 amino acids) for construction by automated solid-phase peptide synthesis, peptide stapling (by, e.g., ruthenium-catalyzed olefin metathesis of artificial amino acids) may be utilized to construct cell-permeable versions that enable gene modulation in whole cells. Fusion of truncated homeodomain helices of Hoxa9 and Pbx1 is expected to yield miniature proteins (˜40 amino acids) capable of binding to the Hoxa9-Pbx1 DNA consensus sequence. To validate DNA-site selectivity we will isolate and purify the fusion proteins from the yeast cell surface by TEV cleavage (cleavage site upstream of N-terminus) and perform in vitro site-selection PCR experiments (SELEX) using random DNA sequences.(25) Using this method we expect to rapidly identify clones capable of selective binding to the Hoxa9-Pbx1 DNA consensus sequence (TGATTTAC). We will then synthesize cell-permeable α-helix stabilized versions of these proteins by solid-phase peptide synthesis followed by ruthenium-catalyzed olefin metathesis to create the staple. Several potential sites of stapling (i, i+7) have been identified at amino acid residues that do not play a role in DNA recognition based on analysis of the Hoxa9-Pbx1-DNA crystal structure. To tether the HFPs to the transcription repressing SID domain, we will use polyethylene glycol (PEG) linkers or glycine-repeats. We will determine the optimal linker and length to identify candidates that simultaneously bind DNA target genes and Sin3 transcription repressor protein in electrophoretic mobility shift assays (EMSA). Cell and nucleus permeability of fluorescein-labeled versions of the fusion protein will be assessed by fluorescence confocal microscopy in myeloid progenitors. To confirm that the stapled fusion proteins bind to the genomic Hoxa9-Pbx1 DNA recognition site, we will treat myeloid progenitors with stapled fusion proteins containing a FLAG tag and perform chromatin immunoprecipitation sequencing (ChIP-Seq) assays. Using this approach we will identify suitable fusion proteins for investigation in AML differentiation.

### Example 4

### Modulation of AML Cell Differentiation in Culture and In Vivo

Using the lysozyme-GFP myeloid progenitor cell assay described in Example 2, the ability of Hoxa9-Pbx1 HFP domains attached to the SID transcription repressor (HFP-TRs) will be tested for their ability to enable myeloid cell differentiation. the fusion proteins (HFP-TRs) will be tested at multiple doses for the ability to induce myeloid differentiation in culture using the estrogen-receptor dependent cell-based assay described in Example 2. Furthermore, changes in the transcript levels of HoxA9 target genes such as Creb1 and Pknox1 will be measured by quantitative real time PCR.(26)

The therapeutic effect of HFP-TRs will be evaluated in a murine model of AML driven by MLL-AF9, a fusion oncoprotein in human AML. We will determine the maximum tolerated dose and the pharmacokinetics of the most promising HFP-TRs to select lead candidates for evaluation in the murine AML model. As MLL-driven AMLs are critically dependent on Hoxa9 activity, HFP-TRs should increase survival of mice transplanted with MLL-AF9 transduced bone marrow cells (˜30-35 day latency).

Given the dependence of myeloid progenitors and AML cells on Hoxa9 and PBX, Hoxa9-PBX DNA recognition sites in the genome that are relevant to the differentiation blockade should be free of histone modifications and accessible to the fusion proteins. If adequate PCR-site selectivity for the Hoxa9-PBX DNA recognition sequence is not observed for HFPs, error-prone PCR can be used to introduce random mutations in focused HFP yeast libraries and directed evolution can be used to achieve desired selectivity for the DNA sequence. Given the lysine and arginine-rich sequence of HFPs, stapled versions of these proteins should be cell-permeable based on previous observations.(27) If sufficient nuclear localization is not achieved then a short nuclear localization sequence (PKKKRKV, SEQ ID NO: 19) will be included during the synthesis of HFPs.(16)

### Example 5

### Exemplary Fusion Proteins

Provided below in Table 1 are domain schematics of the exemplary fusion proteins to illustrate certain embodiments of the invention. The fusion proteins have been tested for activity, which is based upon growth deficit versus non-transduced cells in same well Fusion proteins S3 and 1-10 below contain a C-terminal 3XFLAG (not shown), with a GG linker between the Flag tag and the HFP domain. Fusion protein 10 is a N-terminal 3XFLAG separated by a GG linker from the HFP domain. Each instance of “N” indicates a nuclear localization sequence (DPKKKRKV, SEQ ID NO: 18). “DPA” is the DPA alpha-helix nucleating motif sequence. “Hoxa9-G3-PBX” is the homeodomain fusion protein (HFP) domain comprising the HoxA9 and PBX truncated homeodomains with a GGG linker fusing the two homeodomains and with a DP alpha helix nucleating motif used at the N-terminal side of each Hoxa9 and PBX helices: DPERQVKIWFQNRRMKMKKINGGGDPVSQVSNWFGNKRIRYKKNIG (SEQ ID NO: 34).

TABLE 1

| | SEQ ID | | |
|---|---|---|---|
| Ref. no. | NO: | Domain arrangement | Activity |
| S3 | 11 | 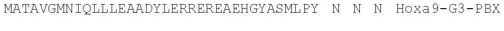 | ✓ |
| 1 | 11 | 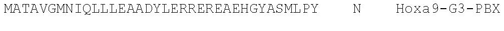 | ✓ |
| 2 | 11 | 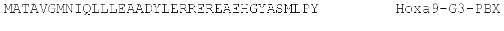 | x |
| 3 | 12 | 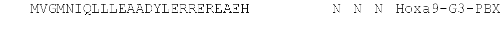 | ✓ |
| 4 | 13 | 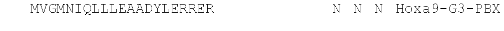 | ✓ |
| 5 | 14 | 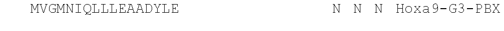 | x |
| 6 | 15 | 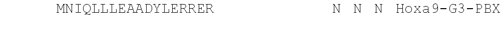 | x |
| 7 | 16 | 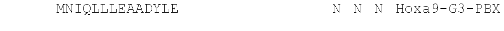 | x |
| 8 | 17 | 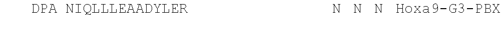 | x |
| 9 | 17 | 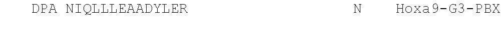 | x |
| 10 | 11 | 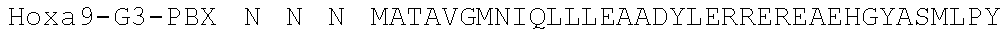 | ✓ |

The amino acid sequences for fusion proteins numbers S3 and 1-10 are provided below. For fusion protein number 10, the SID sequence does not contain the first methionine since the SID sequence does not need the methionine for activity. Since Met is the start codon, it was often placed preceding the start of the HFP domain. In addition, Ala can be incorporated following the Met so that proximity of Met does not interfere with the ucleating activity of DP. Also note that the 3XFLAG tag (not shown) is not an active component of the fusion proteins and is an experimental tool used to enable checking genomic DNA binding sites and assess sequence specificity (for Hox/PBX DNA targets) by ChiP-seq or Chip-PCR to be conducted. The final active versions of the fusion proteins can be with or without the FLAG tag. Other tags in addition to FLAG and 3XFLAG that may be used to determine DNA-target specificity in whole cells are HA-tag or myc-tag.

In Table 1, Ref No. S3=SEQ ID NO: 35; Ref No. 1=SEQ ID NO: 36; Ref No. 2 =SEQ ID NO: 37; Ref No. 3=SEQ ID NO: 38; Ref No. 4=SEQ ID NO: 39; Ref No. 5=SEQ ID NO: 40; Ref No. 6=SEQ ID NO: 41; Ref No. 7=SEQ ID NO: 42; Ref No. 8=SEQ ID NO: 43; Ref No. 9=SEQ ID NO: 44; Ref No. 10=SEQ ID NO: 45. An S3 mutant was also prepared and is SEQ ID NO: 46.

Provided below in Table 2 are domain schematics for an LFN sequence fused to the fusion proteins (LFN-fusion proteins). The fusion proteins will be tested for activity. LFN refers to the N-terminal portion of anthrax toxin lethal factor (LF_{N}) of SEQ ID NO: 25. The LFN-fusion proteins in Table 3 contain a SGGGGS (SEQ ID NO: 57) linker between the LFN domain and the rest of the fusion protein. In Table 3, the domains 3xNLS-LFN has a LFN sequence with an NLS sequence embedded into the LFN sequence. We are yet to test the functionality and cellular localization of these newly-created LFN-HFP proteins.

TABLE 2

| Ref. | |
|---|---|
| No. | Domain arrangement |
| 12 | 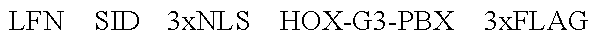 |
| 13 | 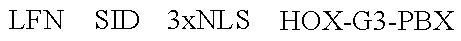 |
| 14 | 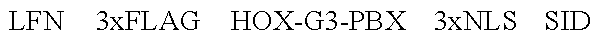 |
| 15 | 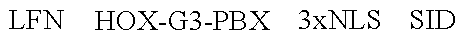 |
| 16 | 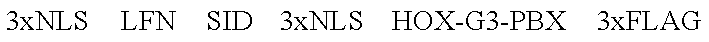 |
| 17 | 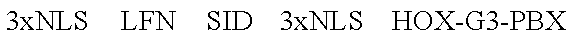 |
| 18 | 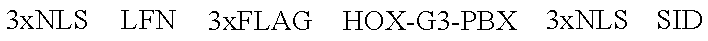 |
| 19 | 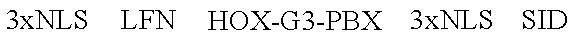 |

In Table 2, Ref No. 12=SEQ ID NO: 47; 13=(SEQ ID NO: 48; 14=SEQ ID NO: 49; 15=SEQ ID NO: 50; 16=SEQ ID NO: 51; 17=SEQ ID NO: 52; 18=SEQ ID NO: 53; 19=SEQ ID NO: 54.

Provided below in Table 3 are exemplary LFN-fusion proteins that can be constructed using full-length homeodomains for HoxA9 and PBX. The NLS domain can be varied to have one NLS to three NLS. The SID sequence can be replaced with KRAB sequence or variant thereof. The linker SGGGGSGGGGS (SEQ ID NO: 58) can be replaced with other linkers of varying lengths and/or composition.

TABLE 3

| Ref. No. | Domain arrangement |
|---|---|
| 20 | 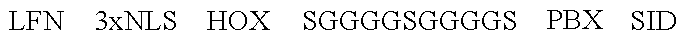 |
| 21 | 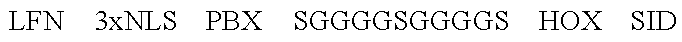 |

### Example 6

### Modulation of AML cell differentiation in culture and in vivo.

To demonstrate the feasibility of homeodomain fusion proteins to target Hoxa9/PBX DNA-binding sites, and modulate transcription, we ectopically expressed non-stapled HFP-SID fusion constructs in the aggressive Hoxa9-Meis1 immortalized AML model. The fusion protein used (SID-NLS-NLS-NLS-GG linker-truncated HoxA9 homeodomain-GGG linker-truncated Pbx homeodomain) was expressed from the S3 construct (see Example 5) that was expressed with a 3XFLAG at the C-terminus (not shown in sequence). The S3 construct was introduced into murine progenitor cells created by transduction of Hoxa9 and Meis1 (to create a murine AML dependent on Hoxa9 and Meis1). The S3 was introduced using a murine stem cell virus (MSCV) retroviral vector containing an IRES GFP. GFP positive cells were sorted at 3 days post-transduction of S3 and cultured for an additional 30 days after which differentiation status was determined by flow cytometry after staining for Mac-1 (CD11b), Gr-1 and FLT3R expression. The control used in the experiments were GFP positive cells created by MSCV IRES GFP vector transduction (empty vector i.e., lacking the S3 construct).

A mutant S3 contruct was also created by replacing amino acids that specifically interact with DNA bases to enable sequence specific-recognition identified using the published crystal structure of the Hoxa9 and PBX bound to DNA. These bases were mutated to Alanine (bold and italicized in Example 5) and the mutant was introduced into Hoxa9-Meis1 murine AML using the MSCV IRES GFP retroviral vector.

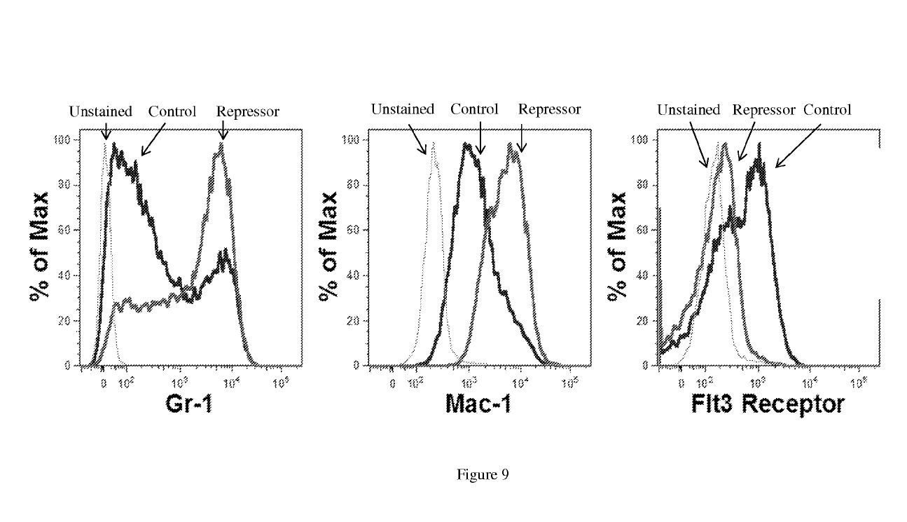

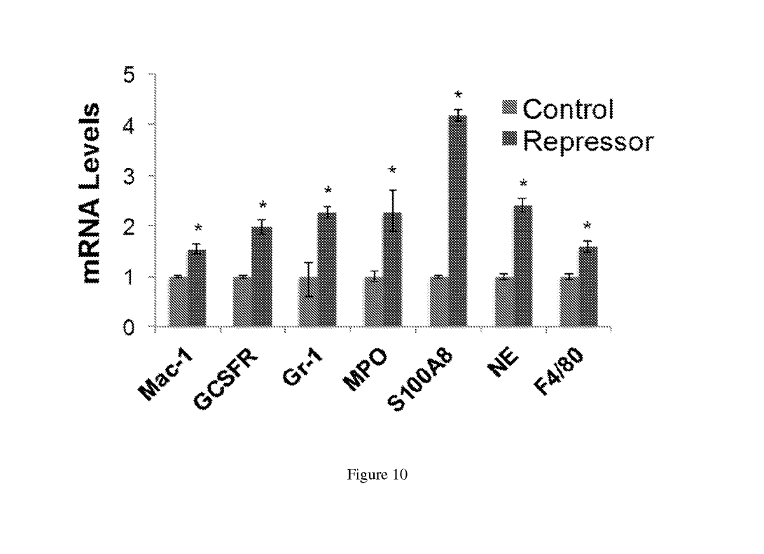

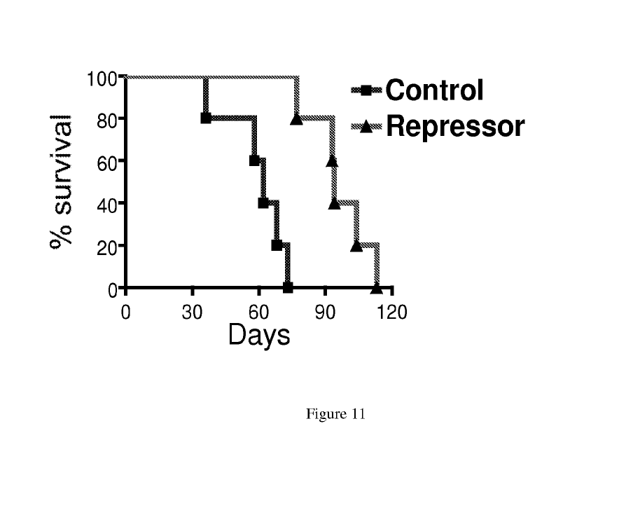

We investigated glycine linkers of varying length (1-4 glycines) to fuse the truncated Hoxa9-PBX helices and observed the 3X glycine linker repressor construct (“Repressor” in FIGS. 9-11) displayed the greatest differentiation-inducing activity with: i) mRNA upregulation of differentiation-specific genes (e.g. S100A8, myeloperoxidase and neutrophil elastase, FIG. 10); ii) increases in cell-surface expression of myeloid differentiation markers (Mac-1 and Gr-1, FIG. 9); and iii) decreased surface expression of Fms-related tyrosine kinase 3 receptor (F1t3R), which is expressed on primitive cells and plays a prominent role in AML progression (FIG. 9). We also confirmed suppression of direct Hoxa9 transcriptional target genes (e.g. SOX4, CD34, FLT3R, FOXP1 and DNAJC10), and demonstrated a mutant construct, in which DNA base-interacting amino acids were mutated to alanine, displays little to no repression of these genes (FIG. 16). Cells transduced with the HFP in an IRES GFP vector showed significant growth defects and were quickly outcompeted by non-transduced GFP-negative cells (HFP-expressing cells decreased to 2% of the population by day 8, while cells transduced with GFP vector control were 80% of the cell population). Mice transplanted with AML cells expressing HFP repressor (FACS sorted) displayed significantly longer latency than vector control (median survival 94 days for repressor versus 62 days for control, p value=0.002, FIG. 12). Analysis of the bone marrow from deceased mice in the repressor group revealed the bulk of AML cells lacked repressor expression (i.e. GFP-negative, most likely due to injection of contaminating non-transduced cells that outcompeted repressor-expressing cells), suggesting our survival benefit may be grossly underestimated. Thus far we have created a total of 25 HFP constructs, of which 9 are active and 16 inactive when transduced into AML cells. Our investigations have identified the minimal length of the SID domain, the necessity of a SV40 nuclear localization sequence (NLS), and tolerance of reverse assembly of the modules (HFP-NLS-SID and SID-NLS-HFP are both active).

Together our results suggest HFPs are capable of targeting Hoxa9-PBX DNA binding sites in whole cells to repress transcription, induce differentiation and increase AML latency in vivo. These results warrant efforts to creating therapeutic versions of the HFPs through use of efficient cell-specific delivery methods.

### Example 7

### Intracellular Delivery of Fusion Proteins

The preliminary results of ectopic retroviral expression of fusion proteins in AML cells suggest our principal focus should be toward achieving the delivery of HFPs to AML cells. However, therapeutic intracellular delivery of proteins has been difficult to realize in general, and the use of cell-penetrating peptides fails to achieve cell-specificity or efficient delivery particularly in hematopoietic cells. Similarly, the use of viral delivery platforms or modified RNAs would be non-specific and difficult to perform in vivo (especially in hematopoietic cells). To address this issue, we aim to leverage a technology which exploits the highly penetrating property of anthrax toxin protein components to efficiently deliver non-anthrax cargo proteins.(23) The anthrax system removes the toxin component, and capitalizes on the pore forming and transporting function of a mutant protective antigen (mPA) to which an scFv or ligand may be attached to target specific cells of interest.(24, 25) As PA specifically transports LF_{N}-containing motifs, cargo HFPs bearing an LF_{N} sequence may be efficiently imported into cells. The versatility of this system is useful for the specific delivery of HFPs to CD33+AML cells (80% of patients) in vivo. As a proof-of-principle, we demonstrated this platform efficiently delivers a LF_{N}-diphtheria toxin fusion (LF_{N}-DTA) protein using wild-type PA (WT-PA) in 5 AML lines (3 murine and 2 human) in vitro and observed cell-killing efficiencies of IC₅₀≈1 picomolar, similar to that observed in non-hematopoietic cell types.

LF_{N}-fusion proteins have been created, expressed and purified recombinant versions from E. Coli. The fusion proteins (8-9 kDa) are attached to the C-terminus of LF_{N}, a 32 kDa protein, and a 3XFlag tag was included at the C-terminus of the HFP. We will test the ability of these LF_{N}-HFPs against the Hoxa9/Meis 1 AML cell line in combination with wild-type PA to determine which constructs most potently inhibit growth and induce differentiation (assessed by flow cytometry of Mac-1 and Gr-1 expression). We will confirm intracellular delivery and nuclear localization by confocal microscopy (using anti-Flag antibodies). We will study LF_{N}-HFPs with 3X NLS added to the N-terminus of LF_{N}. Once we have identified our most potent candidates we will attempt to optimize activity by further systematic alterations. For example, we will explore replacing the Sin3-interacting domain (SID) with a KRAB repressor motif, a more potent repressor.(26) Other modifications may include the shuffling of the order of the various modules (e.g. the 3XNLS, HFP, repressor) to identify the optimal order. Subsequent optimizations will re-investigate the linker length, as the optimal length may have changed from our original versions, and also will create inactive mutants to serve as controls to verify on-target specificity. We expect to quantitatively investigate 15 constructs in each round and perform up to 4 rounds of optimization to identify 2-4 lead LF_{N}-HFPs for mechanistic characterization. Provided below is a flow chart representing path of action for sub-Aim 1A with green and red arrows representing primary and alternate plans, respectively.

In parallel to LF_{N}-fusion protein development, we will develop an mPA-scFv construct for AML-specific delivery using an anti-CD33 human scFv previously reported.(27) We will investigate whether this mPA-scFv can first facilitate delivery of LFN-diphtheria toxin (LF_{N}-DTA) to human AML cells specifically (versus CD33 negative cell lines) and subsequently deliver the lead LFN-fusion proteins.

Once we have identified lead LF_{N}-fusion proteins, we will validate their on-target genomic specificity versus inactive mutant versions. We will perform Q-PCR to demonstrate the upregulation of differentiation-specific transcripts and downregulation of Hoxa9-specific target genes in 5 cell lines as shown in our preliminary results. Furthermore, we will perform ChIP-seq in Hoxa9-Meis1 AML cells to identify the genomic loci to which HFPs bind and confirm TGATTTAT as the consensus recognition sequence. The ChIP-seq data of LF_{N}-HFPs will be compared to reported ChIP-seq data for Hoxa9 and Meis1 to determine the extent of overlap as a measure of on-target specificity. (15) Using this approach the lead LF_{N}-fusion proteins with the desired on-target specificity can be identified.

### REFERENCES

Fenaux, P., Le Deley, M. C., Castaigne, S. Archirabaud, E., Chomienne, C., Link, H., Guerci, A., Duarte, M., Daniel, M. T., Bowen, D., and et al. (1993) Effect of all transretinoic acid in newly diagnosed acute promyelocytic leukemia, Results of a multicenter randomized trial. European APL 91 Group, Blood 82, 3241-3249.
2. Argiropoulos, B., and Humphries, R. K. (2007) Hox genes in hematopoiesis and leukemogenesis, Oncogene 26, 6766-6776.
3. Lawrence, H. J., Sauvageau, G., Humphries, R. K., and Largman, C. (1996) The role of HOX homeobox genes in normal and leukemic hematopoiesis, Stem Cells 14, 281-291.
4. Golub, T. R., Slonim, D. K., Tamayo, P., Huard, C., Gaasenbeek, M., Mesirov, J. P., Coller, H., Loh, M. L., Downing, R., Caligiuri, M. A., Bloomfield, C. D., and Lander, E. S. (1999) Molecular classification of cancer: class discovery and class prediction by gene expression monitoring, Science 286, 531-537.
5. Calvo, K. R., Sykes, D. B., Pasillas, M., and Kamps, M. P. (2000) Hoxa9 immortalizes a granulocyte-macrophage colony-stimulating factor-dependent promyelocyte capable of biphenotypic differentiation to neutrophils or macrophages, independent of enforced meis expression, Mol Cell Biol 20, 3274-3285.
6. Kroon, E., Krosl, J., Thorsteinsdottir, Baban, S., Buchberg, A. M., and Sauvageau, G. (1998) Hoxa9 transforms primary bone marrow cells through specific collaboration with Meis1a but not Pbx1b, EMBO J 17, 3714-3725.
7. Chang, C. P., Brocchieri, L., Shen, W. F., Largman, C., and Cleary, M. L. (1996) Pbx modulation of Hox homeodomain amino-terminal arms establishes different DNA-binding specificities across the Hox locus, Mol Cell Biol 16, 1734-1745.
8. Shen, W. F., Chang, C. P., Rozenfeld, S., Sauvageau, G., Humphries, R. K., Lu, M., Lawrence, H. J., Cleary, M. L., and Largman, C. (1996) Hox homeodomain proteins exhibit selective complex stabilities with Pbx and DNA, Nucleic Acids Res 24, 898-906.
9. Shen, W. F., Rozenfeld, S., Lawrence, H. J., and Largman, C. (1997) The Abd-B-like Hox homeodomain proteins can be subdivided by the ability to form complexes with Pbx1a on a novel DNA target, J Biol Chem 272, 8198-8206.
10. Huang, Y., Sitwala, K., Bronstein, J., Sanders, D., Dandekar, M., Collins, C., Robertson, G., MacDonald, J., Cezard, T., Bilenky, M., Thiessen, N., Zhao, Y., Zeng, T., Hirst, M., Hero, A., Jones, S., and Hess, J. L. (2012) Identification and characterization of Hoxa9 binding sites in hematopoietic cells, Blood 119, 388-398.
11. Moellering, R. E., Cornejo, M., Davis, T. N., Del Bianco, C., Aster, J. C., Blacklow, S. C., Kung, A. L., Gilliland, D. G., Verdine, G. L., and Bradner, J. E. (2009) Direct inhibition of the NOTCH transcription factor complex, Nature 462, 182-188.
12. Kim, Y. W., Grossmann, T. N., and Verdine, G. L. (2011) Synthesis of all-hydrocarbon stapled alpha-helical peptides by ring-closing olefin metathesis, Nat Protoc 6, 761-771.
13. LaRonde-LeBlanc, N. A., and Wolberger, C. (2003) Structure of HoxA9 and Pbx1 bound to DNA: Hox hexapeptide and DNA recognition anterior to posterior, Genes Dev 17, 2060-2072.
14. Chao, G., Lau, W. L., Hackel, B. J., Sazinsky, S. L., Lippow, S. M., and Wittrup, K. D. (2006) Isolating and engineering human antibodies using yeast surface display, Nat Protoc 1, 755-768.
15. Steigemann, W., and Weber, E. (1979) Structure of erythrocruorin in different ligand states refined at 1.4 A resolution, J Mol Biol 127, 309-338.
16. Grzenda, A., Lomberk, G., Zhang, J. S., and Urrutia, R. (2009) Sin3: master scaffold and transcriptional corepressor, Biochim Biophys Acta 1789, 443-450.
17. van Ingen, H., Lasonder, Jansen, J. F., Kaan, A. M., Spronk, C. A., Stunnenberg, H. G., and Vuister, G. W. (2004) Extension of the binding motif of the Sin3 interacting domain of the Mad family proteins, Biochemistry 43, 46-54.
18. Magnenat, L., Blancafort, P., and Barbas, C. F., 3rd. (2004) In vivo selection of combinatorial libraries and designed affinity maturation of polydactyl zinc finger transcription factors for ICAM-1 provides new insights into gene regulation, J Mol Biol 341, 635-649.
19. Ogawa, N., and Biggin, M. D. (2012) High-throughput SELEX determination of DNA sequences bound by transcription factors in vitro, Methods Mol Biol 786, 51-63.
20. Hu, Y. L., Fong, S., Ferrell, C., Largman, C., and Shen, W. F. (2009) HOXA9 modulates its oncogenic partner Meis1 to influence normal hematopoiesis, Mol Cell Biol 29, 5181-5192.
21. Verdine, G. L., and Hilinski, G. J. (2012) All-hydrocarbon stapled peptides as Synthetic Cell-Accessible Mini-Proteins, Drug Discovery Today: Technologies 9, e41-e47.
22. Hodel, M. R., Corbett, A. H., and Hodel, A. E. (2001) Dissection of a nuclear localization J Biol Chem 276, 1317-1325.
23. Shen, W. F., Rozenfeld, S., Kwong, A., Korn yes, L. G., Lawrence, H. J., and Largman, C. (1999) HOXA9 forms triple complexes with PBX2 and MEIS1 in myeloid cells, Mol Cell Biol 19, 3051-3061.
24. Shah, N., and Sukumar, S. (2010) The Hox genes and their roles in oncogenesis, Nat Rev Cancer 10, 361-371.
25. Khan, I., Altman, J. 6., and Licht, J. D. (2012) New strategies in acute myeloid leukemia: redefining prognostic markers to guide therapy, Clin Cancer Res 18, 5163-5171.
26. Hamann, P. R., Hinman, L. M., Beyer, C. F., Lindh, D., Upeslacis, J., Flowers, D. A., and Bernstein, I. (2002) An anti-CD33 antibody-calicheamicin conjugate for treatment of acute myeloid leukemia. Choice of linker, Bioconjug Chem 13, 4046.
27. Petersdorf, S. H., Kopecky, K, J., Slovak, M., Willman, C., Nevill, T., Brandwein, J., Larson, R. A., Erba, H. P., Stiff, P. J., Stuart, R. K., Walter, R. B., Tallman, M. S., Stenke, L., and Appelbaum, F. R. (2013) A phase 3 study of gemtuzurnab ozogamicin during induction and postconsolidation therapy in younger patients with acute myeloid leukemia, Blood 121, 4854-4860.

### Equivalents and Scope

As used in this specification and the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A fusion protein comprising a homeodomain fusion protein (HFP) domain, a nuclear localization sequence (NLS) domain, and a transcription modulator (TM) domain, wherein the homeodomain fusion protein domain comprises a first homeodomain and a second homeodomain.

2-3. (canceled)

4. The fusion protein of claim 1, wherein one of the homeodomains is a HOX homeodomain or a PBX homeodomain.

5. The fusion protein of claim 1, wherein one of the homeodomains is a HoxA9 homeodomain of SEQ ID NO: 5.

6-8. (canceled)

9. The fusion protein of claim 1, wherein one of the homeodomains is a PBX homeodomain of SEQ ID NO: 6.

10-13. (canceled)

14. The fusion protein of claim 1,
wherein the HoxA9 homeodomain comprises the sequence:
X₁RQVX₅X₆WX₈X₉X₁₀RRX₁₃X₁₄X₁₅KX (SEQ ID NO: 1), wherein
X₁ is E or an amino acid capable of cross-linking with another amino acid capable of cross-linking;
each of X₅ and X₁₄ is independently any amino acid residue;
each of X₆, X₉, X₁₀, and X₁₃ is independently any amino acid residue or an amino acid capable of cross-linking with another amino acid capable of cross-linking;
X₈ is F or an amino acid capable of cross-linking with another amino acid capable of cross-linking;
X₁₅ is M or an amino acid capable of cross-linking with another amino acid capable of cross-linking; and
X₁₇ is any amino acid residue; and
wherein the sequence comprises two or three amino acids capable of cross-linking with another amino acid capable of cross-linking.

15-16. (canceled)

17. The fusion protein of any one of the preceding claims claim 1, wherein the HFP domain comprises a HoxA9 homeodomain comprising the sequence:
ERQVKIWFQNRRMKMKKIN (SEQ ID NO: 2).

18. The fusion protein of claim 1, wherein the HFP domain comprises a PBX homeodomain comprising the sequence:
X₁X₂QVSX₆WX₈GX₁₀KRIX₁₄X₁₅KKNIG (SEQ ID NO: 3), wherein
X₁ is V or an amino acid capable of cross-linking with another amino acid capable of cross-linking;
X₂ is any amino acid residue;
X₆ is N or an amino acid capable of cross-linking with another amino acid capable of cross-linking;
X₈ is F or an amino acid capable of cross-linking with another amino acid capable of cross-linking;
X₁₀ is N or an amino acid capable of cross-linking with another amino acid capable of cross-linking;
X₁₄ is R or an amino acid capable of cross-linking with another amino acid capable of cross-linking;
X₁₅ is Y or an amino acid capable of cross-linking with another amino acid capable of cross-linking; and
wherein the sequence comprises two or three amino acids capable of cross-linking with another amino acid capable of cross-linking.

19. (canceled)

20. The fusion protein of claim 1, wherein the HFP domain comprises a PBX homeodomain comprising the sequence:
VSQVSNWFGNKRIRYKKNIG (SEQ ID NO: 4).

21. The fusion protein of claim 1, wherein the HFP domain comprises a first homeodomain that is HoxA9 of SEQ ID NO: 5 or a variant thereof, and a second homeodomain that is PBX of SEQ ID NO: 6 or a variant thereof.

22. The fusion protein of claim 1, wherein the HFP domain comprises a first homeodomain comprising a sequence that is at least about 80% homologous to HoxA9 of SEQ ID NO: 5, and a second homeodomain comprising a sequence that is at least about 80% homologous to PBX of SEQ ID NO: 6.

23-27. (canceled)

28. The fusion protein of claim 1, wherein at least one of the homeodomains comprises an alpha-helix nucleating motif sequence.

29. (canceled)

30. The fusion protein of claim 1, wherein the transcription modulator domain is a transcription repressor domain.

31-35. (canceled)

36. A fusion protein comprising a homeodomain fusion protein (HFP) domain, a nuclear localization sequence (NLS) domain, and a transcription modulator (TM) domain, wherein the homeodomain fusion protein domain comprises first homeodomain comprising a HoxA9 sequence of SEQ ID NO: 2 and a second homeodomain comprising a PBX sequence of SEQ ID NO: 4, and the transcription modulator domain is a transcription repressor (TR) domain.

37-40. (canceled)

41. The fusion protein of claim 1, wherein the fusion protein has one of the following domain arrangements:

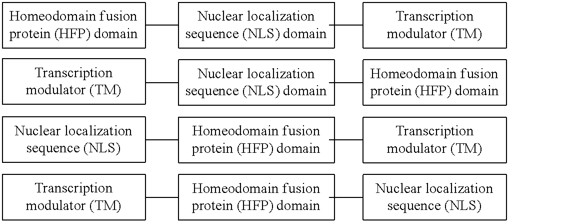

42. The fusion protein of claim 1, wherein the first homeodomain and the second homeodomain are fused using one of the following arrangements:

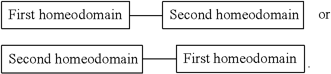

43. (canceled)

44. The fusion protein of claim 1, wherein the nuclear localization sequence (NLS) domain is a NLS or a NLS that is repeated two or three times consecutively within the fusion protein.

45-50. (canceled)

51. The fusion protein of claim 1, wherein the fusion protein comprises an anthrax toxin lethal factor.

52-55. (canceled)

56. The fusion protein of claim 1, wherein the the fusion protein is capable of modulating the transcription of a target gene.

57-58. (canceled)

59. The fusion protein of claim 1, wherein the fusion protein causes cell differentiation.

60-63. (canceled)

64. A polynucleotide encoding the fusion protein of claim 1.

65-68. (canceled)

69. A composition comprising a pore-forming toxin unit and the fusion protein of claim 1.

70-81. (canceled)

82. A pharmaceutical composition comprising the composition of claim 69 and a pharmaceutically acceptable carrier or excipient.

83. A method of treating a disease or disorder, the method comprising administration of the fusion protein of claim 1 to a subject in need thereof.

84-86. (canceled)
